(19) United States
(12) Patent Application Publication
(10) Pub. No.: US 2016/0166556 A1
(43) Pub. Date: Jun. 16, 2016

(54) METHODS OF TREATING PULMONARY HYPERTENSION

(71) Applicant: Gilead Sciences, Inc., Foster City, CA (US)

(72) Inventors: Grant Raymond Budas, Palo Alto, CA (US); John T. Liles, San Jose, CA (US); Dillon Thanh Phan, Pleasanton, CA (US)

(21) Appl. No.: 14/823,457

(22) Filed: Aug. 11, 2015

Related U.S. Application Data
(60) Provisional application No. 62/036,899, filed on Aug. 13, 2014.

(51) Int. Cl. A61K 31/4439; A61K 9/00; A61K 9/20

(52) U.S. Cl. CPC A61K 31/4439; A61K 9/20; A61K 9/0053

(57) ABSTRACT

The present disclosure relates to a method of preventing and/or treating pulmonary vascular disease and/or right ventricular dysfunction, including but not limited to pulmonary hypertension or pulmonary arterial hypertension, comprising administering a therapeutically effective amount of an ASK1 inhibitor.

### CROSS REFERENCE TO RELATED APPLICATIONS

This application claims priority to U.S. Provisional Patent Application Ser. No. 62/036,899, filed Aug. 13, 2014, the entirety of which is incorporated herein by reference.

### FIELD OF THE INVENTION

The present application relates generally to the therapeutics and the methods of using the apoptosis signal regulating kinase 1 (ASK1) inhibitor in treating pulmonary vascular diseases.

### BACKGROUND

Pulmonary arterial hypertension (PAH) is a progressive pulmonary vascular disease that results in death due to right ventricular failure. It is characterized by profound vasoconstriction and pulmonary arterial obstruction that lead to increased pulmonary vascular resistance (PVR), elevated pulmonary artery pressures, right ventricular (RV) dysfunction, and ultimately, right heart failure. Current therapies for PAH target the vasoconstrictive component of this disease. Despite therapies with pulmonary vasodilators, patients with PAH still face a poor prognosis (68% survival at 3 years). There remains an unmet medical need for novel, effective and safe treatments for PAH that directly targets the diseased pulmonary vasculature and the maladaptive remodeling processes in the RV myocardium.

Accordingly, there remains a need to provide new effective pharmaceutical agents to treat pulmonary vascular diseases and/or right ventricular dysfunction.

### SUMMARY

Disclosed herein is a method of treating and/or preventing pulmonary vascular disease and/or right ventricular dysfunction in a patient in need thereof comprises administering to the patient a therapeutically effective amount of an ASK1 inhibitor.

In one aspect, the ASK1 inhibitor is the compound having the structure of formula (I):

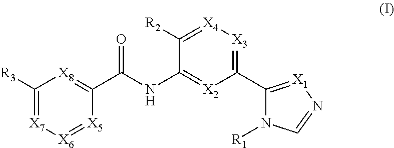

wherein:

R¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to three substituents selected from halo, oxo, alkyl, cycloalkyl, heterocyclyl, aryl, aryloxy, —NO₂, R⁶, —C(O)—R⁶, —OC(O)—R⁶—C(O)—O—R⁶, C(O)—N(R⁶)(R⁷), —OC(O)—N(R⁶)(R⁷), —S—R⁶, —S(═O)—R⁶, —S(═O)₂R⁶, —S(═O)₂—N(R⁶)(R⁷), —S(═O)₂—O—R⁶, —N(R⁶)(R⁷), —N(R⁶)—C(O)—R⁷, —N(R⁶)—C(O)—O—R⁷, —N(R⁶)—C(O)—N(R⁶)(R⁷), —N(R⁶)—S(═O)₂—R⁶, —CN, and —O—R⁶, and wherein the alkyl, cycloalkyl, heterocyclyl, phenyl, and phenoxy are optionally substituted by from one to three substituents selected from alkyl, cycloalkyl, alkoxy, hydroxyl, and halo; wherein R⁶ and R⁷ are independently selected from the group consisting of hydrogen, C₁-C₁₅ alkyl, cycloalkyl, heterocyclyl, aryl, and heteroaryl, all of which are optionally substituted with from one to three substituents selected from halo, alkyl, monoalkylamino, dialkylamino, alkyl amide, aryl amide, heteroaryl amide, —CN, lower alkoxy, —CF₃, aryl, and heteroaryl; or

R⁶ and R⁷ when taken together with the nitrogen to which they are attached form a heterocycle;

R² is hydrogen, halo, cyano, alkoxy, or alkyl optionally substituted by halo;

R³ is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —O—C(O)—R⁶, —O—C(O)—N(R⁶)(R⁷), —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—R⁶, —C(O)—N(R⁶)(R⁷), and —N(R⁶)—S(═O)₂—R⁷, wherein the alkyl, alkoxy, cycloalkyl, aryl, heteroaryl or heterocyclyl is optionally substituted with from one to five substituents selected from halo, oxo, —NO₂, alkyl, haloalkyl, haloalkoxy, —N(R⁶)(R⁷), —C(O)—R⁶, —C(O)—O—R⁶, —C(O)—N(R⁶)(R⁷), —CN, —O—R⁶, cycloalkyl, aryl, heteroaryl and heterocyclyl; with the proviso that the heteroaryl or heterocyclyl moiety includes at least one ring nitrogen atom;

X¹, X², X³, X⁴, X⁵, X⁶, X⁷ and X⁸ are independently C(R⁴) or N, in which each R⁴ is independently hydrogen, alkyl, alkoxy, cycloalkyl, aryl, heteroaryl, heterocyclyl, halo, —NO₂, haloalkyl, haloalkoxy, —CN, —O—R⁶, —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—O—R⁶, —C(O)—N(R⁶)(R⁷), or —N(R⁶)—S(═O)₂—R⁷, wherein the alkyl, cycloalkyl, aryl, heteroaryl, and heterocyclyl is further optionally substituted with from one to five substituents selected from halo, oxo, —NO₂, —CF₃, —O—CF₃, —N(R⁶)(R⁷), —C(O)—R⁶, —C(O)—O—R⁷, —C(O)—N(R⁶)(R⁷), —CN, —O—R⁶; or

X⁵ and X⁶ or X⁶ and X⁷ are joined to provide optionally substituted fused aryl or optionally substituted fused heteroaryl; and

with the proviso that at least one of X², X³, and X⁴ is C(R⁴); at least two of X⁵, X⁶, X⁷, and X⁸ are C(R⁴); and at least one of X², X³, X⁴, X⁵, X⁶, X⁷ and X⁸ is N;

or a pharmaceutically acceptable salt, isomer, or a mixture thereof.

In another aspect, the ASK1 inhibitor is the compound having the structure of formula (II):

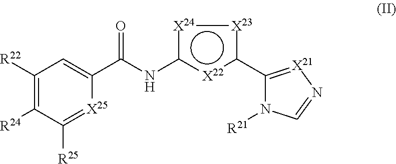

wherein:

R²¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to four substituents selected from the group consisting of halo, hydroxyl, oxo, alkyl, cycloalkyl, heterocyclyl, aryl, aryloxy, NO₂, R²⁶, C(O)R²⁶, OC(O)R²⁶C(O)OR²⁶, C(O)N(R²⁶)(R²⁷), OC(O)N(R²⁶)(R²⁷), SR²⁶, S(═O)R²⁶, S(═O)₂R²⁶, S(═O)₂N(R²⁶)(R²⁷), S(═O)₂OR²⁶, N(R²⁶)(R²⁷), N(R²⁶)C(O)R²⁷, N(R²⁶)C(O)OR²⁷, N(R²⁶)C(O)N(R²⁶)(R²⁷), N(R²⁶)S(═O)₂R²⁶, CN, and OR²⁶, wherein the alkyl, cycloalkyl, heterocyclyl, aryl, and aryloxy are optionally substituted with from one to three substituents selected from alkyl, cycloalkyl, alkoxy, hydroxyl, and halo;

R²⁶ and R²⁷ are independently selected from the group consisting of hydrogen, alkyl, cycloalkyl, heterocyclyl, aryl, and heteroaryl, wherein the alkyl, cycloalkyl, heterocyclyl, aryl, and heteroaryl are optionally substituted with from one to three substituents selected from halo, alkyl, monoalkylamino, dialkylamino, alkyl amide, aryl amide, heteroaryl amide, CN, lower alkoxy, CF₃, aryl, and heteroaryl; or

R²⁶ and R²⁷ when taken together with the nitrogen to which they are attached form a heterocycle;

R²² is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, NO₂, haloalkyl, haloalkoxy, CN, OR²⁶, OC(O)R²⁶, OC(O)N(R²⁶)(R²⁷), SR²⁶, N(R²⁶)(R²⁷), S(═O)R²⁶, S(═O)₂R²⁶, S(═O)₂N(R²⁶)(R²⁷), S(═O)₂OR²⁶, N(R²⁶)C(O)R²⁷, N(R²⁶)C(O)OR²⁷, N(R²⁶)C(O)N(R²⁶)(R²⁷), C(O)R²⁶, C(O)OR²⁶, C(O)N(R²⁶)(R²⁷), and N(R²⁶)S(═O)₂R²⁷, and wherein the alkyl, alkoxy, cycloalkyl, aryl, heteroaryl and heterocyclyl are optionally substituted with one or more substituents selected from halo, oxo, NO₂, alkyl, haloalkyl, haloalkoxy, N(R²⁶)(R²⁷), C(O)R²⁶, C(O)OR²⁶, C(O)N(R²⁶)(R²⁷), CN, OR²⁶, cycloalkyl, aryl, heteroaryl and heterocyclyl; with the proviso that the heteroaryl or heterocyclyl moiety includes at least one ring nitrogen atom;

R²⁴ and R²⁵ are independently hydrogen, halo, cyano, alkyl, alkoxy, or cycloalkyl, wherein the alkyl, alkoxy, and cycloalkyl are optionally substituted by halo or cycloalkyl;

X²¹ and X²⁵ are independently C(R²³) or N, wherein each R²³ is independently hydrogen, halo, alkyl, alkoxy or cycloalkyl, wherein the alkyl and cycloalkyl are optionally substituted with from one to five substituents selected from halo, oxo, CF₃, OCF₃, N(R²⁶)(R²⁷), C(O)R²⁶, C(O)OR²⁷, C(O)N(R²⁶)(R²⁷), CN, and OR²⁶; and

X²², X²³ and X²⁴ are independently C(R²³), N, O, or S; with the proviso that at least one of X²², X²³, and X²⁴ is C(R²³); and only one of X²², X²³, and X²⁴ is O or S;

or a pharmaceutically acceptable salt, isomer, or a mixture thereof.

In additional aspect, the ASK1 inhibitor is the compound of formula (III):

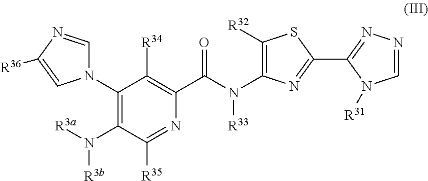

wherein:

R³¹ is C₁-C₃ alkyl or C₃-C₆ cycloalkyl, wherein the alkyl or cycloalkyl is optionally substituted with one to three halogen atoms;

R³² is hydrogen or C₁-C₆ alkyl wherein the alkyl is optionally substituted with halo.

R³³ is hydrogen or C₁-C₃ alkyl;

R³⁴ is hydrogen or C₁-C₃ alkyl;

R³⁵ is hydrogen, C₁-C₃ alkyl, OR^{3a} or —NHR^{3a};

R³⁶ is hydrogen, C₁-C₃ alkyl, C₁-C₃ haloalkyl, or C₃-C₆ cycloalkyl wherein the cycloalkyl is optionally substituted with C₁-C₃ alkyl, C₁-C₃ haloalkyl, or 1 or 2 halogen atoms;

R^{3a} and R^{3b} are independently hydrogen, C₁-C₃ alkyl or R^{3a} and R^{3b} combine with the nitrogen atom to which they are attached to form a four to six member heterocyclic ring optionally containing an oxygen or a nitrogen atom in the ring;

or a pharmaceutically acceptable salt, isomer, or mixture thereof.

In another aspect, the present application provides a method for treating the pulmonary vascular disease such as pulmonary hypertension and pulmonary arterial hypertension. The application also provides a method of treating or preventing right ventricle failure, treating or preventing narrowing or restricting pulmonary arteries, or treating or improving PAH symptoms comprising administering an effective amount of ASK1 inhibitor.

In further aspect, the ASK1 inhibitor is a compound selected from the group consisting of 3-(4-cyclopropyl-1H-imidazol-1-yl)-N-(6-(4-isopropyl-4H-1,2,4-triazol-3-yl)pyridin-2-yl)benzamide, 3-(4-cyclopropyl-1H-imidazol-1-yl)-N-(6-(4-cyclopropyl-4H-1,2,4-triazol-3-yl)pyridin-2-yl)-4-methylbenzamide, 5-(4-cyclopropyl-1H-imidazol-1-yl)-2-fluoro-N-(6-(4-isopropyl-4H-1,2,4-triazol-3-yl)pyridin-2-yl)-4-methylbenzamide, 4-(4-cyclopropyl-1H-imidazol-1-yl)-N-(3-(4-cyclopropyl-4H-1,2,4-triazol-3-yl)phenyl)picolinamide, and (S)-5-(4-cyclopropyl-1H-imidazol-1-yl)-2-fluoro-4-methyl-N-(6-(4-(1,1,1-trifluoropropan-2-yl)-4H-1,2,4-triazol-3-yl)pyridin-2-yl)benzamide, or a pharmaceutically acceptable salt thereof. Also, the ASK1 inhibitor is administered at a dose of between 1 to 100 mg or between 1 to 30 mg. Additionally, the ASK1 inhibitor is administered orally, nasally, topically, or parenterally. Moreover, the method of treating pulmonary vascular disease and/or right ventricular dysfunction comprises administering the ASK1 inhibitor and one or more therapeutic agent.

In the methods provided herein, the ASK1 inhibitor may be administered as a pharmaceutical composition. In some instance, the pharmaceutical composition is a tablet. Accordingly, provided herein is a pharmaceutical composition comprising a therapeutically effective amount of an ASK1 inhibitor and at least one pharmaceutically acceptable carrier.

### BRIEF DESCRIPTION OF THE DRAWINGS

FIG. 1 shows the levels of phosphorylated p38 (phosphor-p38) in the rat right ventricle (RV) in the presence of vehicle or Compound 3 (0.3 mg/kg, 1.0 mg/kg, 3.0 mg/kg, or 10.0 mg/kg) as analyzed by Western blots (panel A). IP90 was used as a loading control. The Western blot signals of phosphorylated p38 (p-p38) were normalized to those of IP90 (panel B). * p<0.05 vs. vehicle; # p<0.05 vs. auranofin (unpaired t-test).

FIG. 2 shows pulmonary hemodynamics and RV hypertrophy in the Sugen/Hypoxia (Su/Hx) model at 4 weeks following disease induction with Su/Hx: systolic pulmonary arterial pressure (PAP) (panel A), mean PAP (panel B), RV hypertrophy (RV/LV)(panel C), and circulating plasma levels of B-type natriuretic peptide (BNP) (panel D). The Su/Hx-rats were treated with vehicle, Compound 4 (0.1% and 0.2% in chow), or sildenafil (30 mg/kg, twice daily) (n=9-10). The control rats did not receive Su/Hx, Compound 4, or sildenafil (control) (n=5). * p<0.05 vs. control, and † p<0.05 vs. vehicle (ANOVA followed by Newman-Keuls multiple comparison test). ̂ p<0.05 Compound 3 vs. vehicle (t-test).

FIG. 3 shows the percent of completely muscularized pulmonary arteries (diameter of 10 to 50 μm) in the Sugen/Hypoxia (Su/Hx) model. A total of 100 intra-acinar pulmonary arterioles per rat were categorized as nonmuscularized (elastin without apparent smooth muscle), partially muscularized (incomplete medial layer of smooth muscle), or completely muscularized (concentric medial layer of smooth muscle). The Su/Hx-rats were treated with vehicle, Compound 4 (0.1% and 0.2% in chow), or sildenafil (30 mg/kg, twice daily) (n=9-10). The control rats did not receive Su/Hx, Compound 4, or sildenafil (control) (n=5). * p<0.05 vs. control; † p<0.05 vs. vehicle (ANOVA followed by Newman-Keuls multiple comparison test).

### DETAILED DESCRIPTION

As used in the present specification, the following terms and phrases are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

As referred to herein, an “ASK1 inhibitor” may be any agent that is capable of inactivating an apoptosis signal regulating kinase 1 (ASK1) protein. The agent may be a chemical compound or biological molecule (e.g., a protein or antibody). The ASK1 protein activity may be measured by several different methods. For example, the activity of an ASK1 protein may be determined based on the ability of the ASK1 protein to phosphorylate a substrate protein. Methods for identifying an ASK1 inhibitor are known (see, e.g., U.S. Patent Application Publication Nos. 2007/0276050, 2011/0009410, 2013/0197037, 2013/0197037, and 2014/0179663 all of which are incorporated herein by reference in their entirety). Exemplary ASK1 substrate proteins include MAPKK3, MAPKK4, MAPKK6, MAPKK7, or fragments thereof. The ASK1 protein activity may be measured by the phosphorylation level of the ASK1 protein, for example, the phosphorylation level of a threonine residue in the ASK1 protein corresponding to threonine 838 (T₈₃₈) of a human full-length ASK1 protein or threonine 845 (T₈₄₅) of a mouse full-length ASK1 protein. For example, where the ASK1 protein comprises a full-length human ASK1 protein sequence, an ASK1 inhibitor may attenuate phosphorylation of T₈₃₈ in the full-length human ASK1 protein sequence. A site-specific antibody against human ASK1 T₈₃₈ or mouse ASK1 T₈₄₅ may be used to detect the phosphorylation level.

The term “pharmaceutically acceptable salt” refers to salts of pharmaceutical compounds e.g. compounds of formulae (I), (IA), (II), or (III) that retain the biological effectiveness and properties of the underlying compound, and which are not biologically or otherwise undesirable. There are acid addition salts and base addition salts. Pharmaceutically acceptable acid addition salts may be prepared from inorganic and organic acids. Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge et al. (J. Pharm. Sci. 1977; 66 (1):1-19).

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)).

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The terms “prevention” or “preventing” mean any treatment of a disease or condition (e.g. pulmonary vascular disease) that stops clinical symptoms of the disease or condition from developing. Compounds may, in some embodiments, be administered to a subject (including a human) who is at risk or has a family history of the disease or condition.

The terms “treatment” or “treating” mean administering a compound or pharmaceutically acceptable salt, isomer, or a mixture thereof described herein for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

“Subject” or “patient” refer to an animal, such as a mammal (including a human), that has been or will be the object of treatment, observation, or experiment. The methods described herein may be useful in human therapy and/or veterinary applications. In some embodiments, the subject is a mammal. In one embodiment, the subject is a human. “Human in need thereof” or “patient in need thereof” refer to a human or a patient, respectively, who may have or is suspect to have diseases, or disorders, or conditions that would benefit from certain treatment. As used herein, the terms “disease,” “disorder,” or “condition” are interchangeable.

A dash (“-”) that is not between two letters or symbols is used to indicate a point of attachment for a substituent. For example, —CONH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” or “Cᵤ-Cᵥ” indicates that the following group has from u to v carbon atoms. For example, “C₁₋₆ alkyl” or “C₁-C₆ alkyl” indicates that the alkyl group has from 1 to 6 carbon atoms.

The term “alkyl” refers to a monoradical branched or unbranched saturated hydrocarbon chain having from 1 to 20 carbon atoms, or from 1 to 15 carbon atoms, or from 1 to 10 carbon atoms, or from 1 to 8 carbon atoms, or from 1 to 6 carbon atoms, or from 1 to 4 carbon atoms. This term is exemplified by groups such as methyl, ethyl, n-propyl, iso-propyl, n-butyl, iso-butyl, t-butyl, n-hexyl, n-decyl, tetradecyl, and the like. The term “substituted alkyl” refers to: (1) an alkyl group as defined above, having 1, 2, 3, 4 or 5 substituents, (in some embodiments, 1, 2 or 3 substituents) selected from the group consisting of alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2; or (2) an alkyl group as defined above that is interrupted by 1-10 atoms (e.g. 1, 2, 3, 4 or 5 atoms) independently chosen from oxygen, sulfur and NR^{a}, where R^{a} is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2; or (3) an alkyl group as defined above that has both 1, 2, 3, 4 or 5 substituents as defined above and is also interrupted by 1-10 atoms (e.g. 1, 2, 3, 4 or 5 atoms) as defined above. The term “lower alkyl” refers to a monoradical branched or unbranched saturated hydrocarbon chain having 1, 2, 3, 4, 5 or 6 carbon atoms. The exemplified group includes but is not limited to methyl, ethyl, n-propyl, iso-propyl, n-butyl, iso-butyl, t-butyl, n-hexyl, and the like. The term “substituted lower alkyl” refers to lower alkyl as defined above having 1 to 5 substituents (in some embodiments, 1, 2 or 3 substituents), as defined for substituted alkyl or a lower alkyl group as defined above that is interrupted by 1, 2, 3, 4 or 5 atoms as defined for substituted alkyl or a lower alkyl group as defined above that has both 1, 2, 3, 4 or 5 substituents as defined above and is also interrupted by 1, 2, 3, 4 or 5 atoms as defined above.

The term “alkylene” refers to a diradical of a branched or unbranched saturated hydrocarbon chain, in some embodiments, having from 1 to 20 carbon atoms (e.g. 1-10 carbon atoms or 1, 2, 3, 4, 5 or 6 carbon atoms). This term is exemplified by groups such as methylene (—CH₂—), ethylene (—CH₂CH₂—), the propylene isomers (e.g., —CH₂CH₂CH₂— and —CH(CH₃)CH₂—), and the like. The term “lower alkylene” refers to a diradical of a branched or unbranched saturated hydrocarbon chain, in some embodiments, having 1, 2, 3, 4, 5 or 6 carbon atoms. The term “substituted alkylene” refers to an alkylene group as defined above having 1 to 5 substituents (in some embodiments, 1, 2 or 3 substituents) as defined for substituted alkyl.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “aralkyloxy” refers to the group —O-aralkyl. “Optionally substituted aralkyloxy” refers to an optionally substituted aralkyl group covalently linked to an optionally substituted alkylene group. Such aralkyl groups are exemplified by benzyloxy, phenylethyloxy, and the like.

The term “alkenyl” refers to a monoradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds. In some embodiments, alkenyl groups include ethenyl (or vinyl, i.e. —CH═CH₂), 1-propylene (or allyl, i.e. —CH₂CH═CH₂), isopropylene (—C(CH₃)═CH₂), and the like. The term “lower alkenyl” refers to alkenyl as defined above having from 2 to 6 carbon atoms. The term “substituted alkenyl” refers to an alkenyl group as defined above having 1 to 5 substituents (in some embodiments, 1, 2 or 3 substituents) as defined for substituted alkyl. The term “alkenylene” refers to a diradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds.

The term “alkynyl” refers to a monoradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds. In some embodiments, alkynyl groups include ethynyl (—C≡CH), propargyl (or propynyl, i.e. —C≡CCH₃), and the like. The term “substituted alkynyl” refers to an alkynyl group as defined above having 1 to 5 substituents (in some embodiments, 1, 2 or 3 substituents) as defined for substituted alkyl.

The term “alkynylene” refers to a diradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds.

The term “hydroxy” or “hydroxyl” refers to a group —OH.

The term “alkoxy” refers to the group R—O—, where R is alkyl or —Y—Z, in which Y is alkylene and Z is alkenyl or alkynyl, where alkyl, alkenyl and alkynyl are as defined herein. In some embodiments, alkoxy groups are alkyl-O— and includes, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like. The term “lower alkoxy” refers to the group R—O— in which R is optionally substituted lower alkyl. This term is exemplified by groups such as methoxy, ethoxy, n-propoxy, iso-propoxy, n-butoxy, iso-butoxy, t-butoxy, n-hexyloxy, and the like. The term “substituted alkoxy” refers to the group R—O—, where R is substituted alkyl or —Y—Z, in which Y is substituted alkylene and Z is substituted alkenyl or substituted alkynyl, where substituted alkyl, substituted alkenyl and substituted alkynyl are as defined herein.

The term “C₁₋₃haloalkyl” refers to an alkyl group having from 1 to 3 carbon atoms covalently bonded to from 1 to 7, or from 1 to 6, or from 1 to 3, halogen(s), where alkyl and halogen are defined herein. In some embodiments, C₁₋₃ haloalkyl includes, by way of example, trifluoromethyl, difluoromethyl, fluoromethyl, 2,2,2-trifluoroethyl, 2,2-difluoroethyl, 2-fluoroethyl, 3,3,3-trifluoropropyl, 3,3-difluoropropyl, 3-fluoropropyl.

The term “C₁₋₃ hydroxyalkyl” refers to an alkyl group having a carbon atom covalently bonded to a hydroxy, where alkyl and hydroxy are defined herein. In some embodiments, C₁₋₃ hydroxyalkyl includes, by way of example, 2-hydroxyethyl.

The term “C₁₋₃ cyanoalkyl” refers to an alkyl group having a carbon atom covalently bonded to a cyano, where alkyl and cyano are defined herein. In some embodiments, C₁₋₃ cyanoalkyl includes, by way of example, 2-cyanoethyl.

The term “cycloalkyl” refers to cyclic alkyl groups of from 3 to 20 carbon atoms, or from 3 to 10 carbon atoms, having a single cyclic ring or multiple condensed rings. Such cycloalkyl groups include, by way of example, single ring structures such as cyclopropyl, cyclobutyl, cyclopentyl, cyclooctyl and the like or multiple ring structures such as adamantanyl and bicyclo[2.2.1]heptanyl or cyclic alkyl groups to which is fused an aryl group, for example indanyl, and the like, provided that the point of attachment is through the cyclic alkyl group.

The term “cycloalkenyl” refers to cyclic alkyl groups of from 3 to 20 carbon atoms having a single cyclic ring or multiple condensed rings and having at least one double bond and in some embodiments, from 1 to 2 double bonds.

The terms “substituted cycloalkyl” and “substituted cycloalkenyl” refer to cycloalkyl or cycloalkenyl groups having 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. The term “substituted cycloalkyl” also includes cycloalkyl groups wherein one or more of the annular carbon atoms of the cycloalkyl group has an oxo group bonded thereto. In addition, a substituent on the cycloalkyl or cycloalkenyl may be attached to the same carbon atom as the attachment of the substituted cycloalkyl or cycloalkenyl to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “cycloalkoxy” refers to the group cycloalkyl-O—. The term “substituted cycloalkoxy” refers to the group substituted cycloalkyl-O—.

The term “cycloalkenyloxy” refers to the group cycloalkenyl-O—. The term “substituted cycloalkenyloxy” refers to the group substituted cycloalkenyl-O—.

The term “aryl” refers to an aromatic carbocyclic group of 6 to 20 carbon atoms having a single ring (e.g., phenyl) or multiple rings (e.g., biphenyl) or multiple condensed (fused) rings (e.g., naphthyl, fluorenyl and anthryl). In some embodiments, aryls include phenyl, fluorenyl, naphthyl, anthryl, and the like.

Unless otherwise constrained by the definition for the aryl substituent, such aryl groups can optionally be substituted with 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “heterocyclyl,” “heterocycle,” or “heterocyclic” refers to a monoradical saturated group having a single ring or multiple condensed rings, having from 1 to 40 carbon atoms and from 1 to 10 hetero atoms, and from 1 to 4 heteroatoms, selected from nitrogen, sulfur, phosphorus, and/or oxygen within the ring. In some embodiments, the heterocyclyl,” “heterocycle,” or “heterocyclic” group is linked to the remainder of the molecule through one of the heteroatoms within the ring.

Unless otherwise constrained by the definition for the heterocyclic substituent, such heterocyclic groups can be optionally substituted with 1 to 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. In addition, a substituent on the heterocyclic group may be attached to the same carbon atom as the attachment of the substituted heterocyclic group to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Examples of heterocyclics include tetrahydrofuranyl, morpholino, piperidinyl, and the like.

The term “heterocycloxy” refers to the group —O-heterocyclyl.

The term “heteroaryl” refers to a group comprising single or multiple rings comprising 1 to 15 carbon atoms and 1 to 4 heteroatoms selected from oxygen, nitrogen and sulfur within at least one ring. The term “heteroaryl” is generic to the terms “aromatic heteroaryl” and “partially saturated heteroaryl”. The term “aromatic heteroaryl” refers to a heteroaryl in which at least one ring is aromatic, regardless of the point of attachment. Examples of aromatic heteroaryls include pyrrole, thiophene, pyridine, quinoline, and pteridine. The term “partially saturated heteroaryl” refers to a heteroaryl having a structure equivalent to an underlying aromatic heteroaryl which has had one or more double bonds in an aromatic ring of the underlying aromatic heteroaryl saturated. Examples of partially saturated heteroaryls include dihydropyrrole, dihydropyridine, chroman, 2-oxo-1,2-dihydropyridin-4-yl, and the like.

Unless otherwise constrained by the definition for the heteroaryl substituent, such heteroaryl groups can be optionally substituted with 1 to 5 substituents (in some embodiments, 1, 2 or 3 substituents) selected from the group consisting alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Such heteroaryl groups can have a single ring (e.g., pyridyl or furyl) or multiple condensed rings (e.g., indolizinyl, benzothiazole or benzothienyl). Examples of nitrogen heterocyclyls and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-alkoxy-nitrogen containing heteroaryl compounds.

The term “heteroaryloxy” refers to the group heteroaryl-O—.

The term “amino” refers to the group —NH₂. The term “substituted amino” refers to the group —NRR where each R is independently selected from the group consisting of hydrogen, alkyl, cycloalkyl, aryl, heteroaryl and heterocyclyl provided that both R groups are not hydrogen or a group —Y—Z, in which Y is optionally substituted alkylene and Z is alkenyl, cycloalkenyl or alkynyl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkyl amine” refers to R—NH₂ in which R is optionally substituted alkyl. The term “dialkyl amine” refers to R—NHR in which each R is independently an optionally substituted alkyl. The term “trialkyl amine” refers to NR₃ in which each R is independently an optionally substituted alkyl.

The term “cyano” refers to the group —CN.

The term “azido” refers to a group

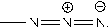

The term “keto” or “oxo” refers to a group ═O.

The term “carboxy” refers to a group —C(O)—OH.

The term “ester” or “carboxyester” refers to the group —C(O)OR, where R is alkyl, cycloalkyl, aryl, heteroaryl or heterocyclyl, which may be optionally further substituted by alkyl, alkoxy, halogen, CF₃, amino, substituted amino, cyano or —S(O)ₙR^{a}, in which R^{a} is alkyl, aryl or heteroaryl and n is 0, 1 or 2.

The term “acyl” denotes the group —C(O)R, in which R is hydrogen, alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “carboxyalkyl” refers to the groups —C(O)O-alkyl or —C(O)O— cycloalkyl, where alkyl and cycloalkyl are as defined herein, and may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aminocarbonyl” refers to the group —C(O)NRR where each R is independently hydrogen, alkyl, cycloalkyl, aryl, heteroaryl, or heterocyclyl, or where both R groups are joined to form a heterocyclic group (e.g., morpholino). Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acyloxy” refers to the group —OC(O)—R, in which R is alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acylamino” refers to the group —NRC(O)R where each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkoxycarbonylamino” refers to the group —N(R^{d})C(O)OR in which R is alkyl and R^{d} is hydrogen or alkyl. Unless otherwise constrained by the definition, each alkyl may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aminocarbonylamino” refers to the group —NR^{c}C(O)NRR, wherein R^{c} is hydrogen or alkyl and each R is hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “thiol” refers to the group —SH. The term “thiocarbonyl” refers to a group ═S. The term “alkylthio” refers to the group —S-alkyl. The term “substituted alkylthio” refers to the group —S-substituted alkyl. The term “heterocyclylthio” refers to the group —S-heterocyclyl. The term “arylthio” refers to the group —S-aryl. The term “heteroarylthiol” refers to the group —S-heteroaryl wherein the heteroaryl group is as defined above including optionally substituted heteroaryl groups as also defined above. The term “sulfoxide” refers to a group —S(O)R, in which R is alkyl, cycloalkyl, heterocyclyl, aryl or heteroaryl. The term “substituted sulfoxide” refers to a group —S(O)R, in which R is substituted alkyl, substituted cycloalkyl, substituted heterocyclyl, substituted aryl or substituted heteroaryl, as defined herein. The term “sulfone” refers to a group —S(O)₂R, in which R is alkyl, cycloalkyl, heterocyclyl, aryl or heteroaryl. Also, the term “substituted sulfone” refers to a group —S(O)₂R, in which R is substituted alkyl, substituted cycloalkyl, substituted heterocyclyl, substituted aryl or substituted heteroaryl, as defined herein.

The term “aminosulfonyl” refers to the group —S(O)₂NRR, wherein each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, where R^{a} is alkyl, aryl or heteroaryl and n is 0, 1 or 2.

The term “hydroxyamino” refers to the group —NHOH. The term “alkoxyamino” refers to the group —NHOR in which R is optionally substituted alkyl.

The term “halogen” or “halo” refers to fluoro, bromo, chloro and iodo.

“Optional” or “optionally” means that the subsequently described event or circumstance may or may not occur, and that the description includes instances where said event or circumstance occurs and instances in which it does not.

A “substituted” group includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (moiety) is described herein as being attached to a second group and the site of attachment is not explicit, the given group may be attached at any available site of the given group to any available site of the second group. For example, a “lower alkyl-substituted phenyl”, where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to -substituted aryl-(substituted aryl)-substituted aryl.

ASK1 Inhibitors

An ASK1 inhibitor for use in the methods and pharmaceutical compositions disclosed herein may be any chemical compound or biological molecule (e.g., a protein or antibody) capable of inactivating apoptosis signal regulating kinase 1 (ASK1) protein. ASK1 inhibitors for use in the methods described herein are known (see, e.g., U.S. Patent Application Publication Nos. 2011/0009410, 2013/0197037, 2013/0197037, 2014/0179663, and 2014/0018370, all of which are incorporated herein by reference in their entirety) and/or can be identified via known methods (see, e.g., U.S. Patent Application Publication Nos. 2007/0276050 and 2011/0009410, which are incorporated herein by reference in their entirety).

In certain embodiments, the ASK1 inhibitor is a compound having the structure of formula (I):

wherein:

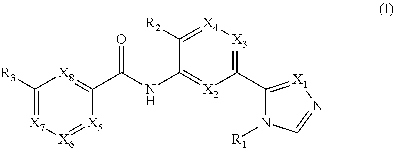

R¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to three substituents selected from halo, oxo, alkyl, cycloalkyl, heterocyclyl, aryl, aryloxy, —NO₂, R⁶, —C(O)—R⁶, —OC(O)—R⁶—C(O)—O—R⁶, C(O)—N(R⁶)(R⁷), —OC(O)—N(R⁶)(R⁷), —S—R⁶, —S(═O)—R⁶, —S(═O)₂R⁶, —S(═O)₂—N(R⁶)(R⁷), —S(═O)₂—O—R⁶, —N(R⁶)(R⁷), —N(R⁶)—C(O)—R⁷, —N(R⁶)—C(O)—O—R⁷, —N(R⁶)—C(O)—N(R⁶)(R⁷), —N(R⁶)—S(═O)₂—R⁶, —CN, and —O—R⁶, and wherein the alkyl, cycloalkyl, heterocyclyl, phenyl, and phenoxy are optionally substituted by from one to three substituents selected from alkyl, cycloalkyl, alkoxy, hydroxyl, and halo; wherein R⁶ and R⁷ are independently selected from the group consisting of hydrogen, alkyl, cycloalkyl, heterocyclyl, aryl, and heteroaryl, all of which are optionally substituted with from one to three substituents selected from halo, alkyl, monoalkylamino, dialkylamino, alkyl amide, aryl amide, heteroaryl amide, —CN, lower alkoxy, —CF₃, aryl, and heteroaryl; or

R⁶ and R⁷ when taken together with the nitrogen to which they are attached form a heterocycle;

R² is hydrogen, halo, cyano, alkoxy, or alkyl optionally substituted by halo;

R³ is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —O—C(O)—R⁶, —O—C(O)—N(R⁶)(R⁷), —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—R⁶, —C(O)—N(R⁶)(R⁷), and —N(R⁶)—S(═O)₂—R⁷, wherein the alkyl, alkoxy, cycloalkyl, aryl, heteroaryl or heterocyclyl is optionally substituted with from one to five substituents selected from halo, oxo, —NO₂, alkyl, haloalkyl, haloalkoxy, —N(R⁶)(R⁷), —C(O)—R⁶, —C(O)—O—R⁶, —C(O)—N(R⁶)(R⁷), —CN, —O—R⁶, cycloalkyl, aryl, heteroaryl and heterocyclyl; with the proviso that the heteroaryl or heterocyclyl moiety includes at least one ring nitrogen atom;

X¹, X², X³, X⁴, X⁵, X⁶, X⁷ and X⁸ are independently C(R⁴) or N, in which each R⁴ is independently hydrogen, alkyl, alkoxy, cycloalkyl, aryl, heteroaryl, heterocyclyl, halo, —NO₂, haloalkyl, haloalkoxy, —CN, —O—R⁶, —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—O—R⁶, —C(O)—N(R⁶)(R⁷), or —N(R⁶)—S(═O)₂—R⁷, wherein the alkyl, cycloalkyl, aryl, heteroaryl, and heterocyclyl is further optionally substituted with from one to five substituents selected from halo, oxo, —NO₂, —CF₃, —O—CF₃, —N(R⁶)(R⁷), —C(O)—R⁶, —C(O)—O—R⁷, —C(O)—N(R⁶)(R⁷), —CN, —O—R⁶; or

X⁵ and X⁶ or X⁶ and X⁷ are joined to provide optionally substituted fused aryl or optionally substituted fused heteroaryl; and

with the proviso that at least one of X², X³, and X⁴ is C(R⁴); at least two of X⁵, X⁶, X⁷, and X⁸ are C(R⁴); and at least one of X², X³, X⁴, X⁵, X⁶, X⁷ and X⁸ is N;

or a pharmaceutically acceptable salt, isomer, or a mixture thereof.

In certain embodiments, the compound of formula (I) has the structure of formula (IA):

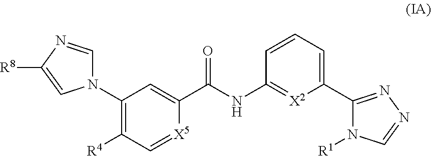

wherein:

R¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to three substituents selected from halo, oxo, alkyl, cycloalkyl, heterocyclyl, aryl, aryloxy, —NO₂, R⁶, —C(O)—R⁶, —OC(O)—R⁶—C(O)—O—R⁶, C(O)—N(R⁶)(R⁷), —OC(O)—N(R⁶)(R⁷), —S—R⁶, —S(═O)—R⁶, —S(═O)₂R⁶, —S(═O)₂—N(R⁶)(R⁷), —S(═O)₂—O—R⁶, —N(R⁶)(R⁷), —N(R⁶)—C(O)—R⁷, —N(R⁶)—C(O)—O—R⁷, —N(R⁶)—C(O)—N(R⁶)(R⁷), —N(R⁶)—S(═O)₂—R⁶, —CN, and —O—R⁶, and wherein the alkyl, cycloalkyl, heterocyclyl, phenyl, and phenoxy are optionally substituted by from one to three substituents selected from alkyl, cycloalkyl, alkoxy, hydroxyl, and halo; wherein R⁶ and R⁷ are independently selected from the group consisting of hydrogen, alkyl, cycloalkyl, heterocyclyl, aryl, and heteroaryl, all of which are optionally substituted with from one to three substituents selected from halo, alkyl, monoalkylamino, dialkylamino, alkyl amide, aryl amide, heteroaryl amide, —CN, lower alkoxy, —CF₃, aryl, and heteroaryl; or

R⁶ and R⁷ when taken together with the nitrogen to which they are attached form a heterocycle;

R⁸ is hydrogen, alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —O—C(O)—R⁶, —O—C(O)—N(R⁶)(R⁷), —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—R⁶, —C(O)—N(R⁶)(R⁷), and —N(R⁶)—S(═O)₂—R⁷, wherein the alkyl, alkoxy, cycloalkyl, aryl, heteroaryl or heterocyclyl is optionally substituted with from one to five substituents selected from halo, oxo, —NO₂, alkyl, haloalkyl, haloalkoxy, —N(R⁶)(R⁷), —C(O)—R⁶, —C(O)—O—R⁶, —C(O)—N(R⁶)(R⁷), —CN, —O—R⁶, cycloalkyl, aryl, heteroaryl and heterocyclyl; with the proviso that the heteroaryl or heterocyclyl moiety includes at least one ring nitrogen atom;

X² and X⁵ are independently C(R⁴) or N; and

each R⁴ is independently hydrogen, alkyl, alkoxy, cycloalkyl, aryl, heteroaryl, heterocyclyl, halo, —NO₂, haloalkyl, haloalkoxy, —CN, —O—R⁶, —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—O—R⁶, —C(O)—N(R⁶)(R⁷), or —N(R⁶)—S(═O)₂—R⁷, wherein the alkyl, cycloalkyl, aryl, heteroaryl, and heterocyclyl is further optionally substituted with from one to five substituents selected from halo, oxo, —NO₂, —CF₃, —O—CF₃, —N(R⁶)(R⁷), —C(O)—R⁶, —C(O)—O—R⁷, —C(O)—N(R⁶)(R⁷), —CN, and —O—R⁶;

with the proviso that at least one of X² and X⁵ is N;

or a pharmaceutically acceptable salt, isomer, or a mixture thereof.

Exemplary compounds of Formula (I) and (IA) for use in the methods and pharmaceutical compositions described herein can be found in U.S. Patent Application Publication No. 2011/0009410, which is incorporated herein by reference in its entirety.

In certain embodiments, the ASK1 inhibitor is a compound of formula (II):

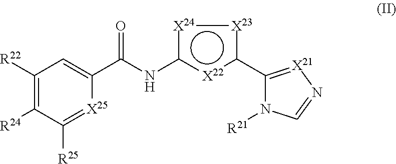

wherein:

R²¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to four substituents selected from the group consisting of halo, hydroxyl, oxo, alkyl, cycloalkyl, heterocyclyl, aryl, aryloxy, NO₂, R²⁶, C(O)R²⁶, OC(O)R²⁶C(O)OR²⁶, C(O)N(R²⁶)(R²⁷), OC(O)N(R²⁶)(R²⁷), SR²⁶, S(═O)R²⁶, S(═O)₂R²⁶, S(═O)₂N(R²⁶)(R²⁷), S(═O)₂OR²⁶, N(R²⁶)(R²⁷), N(R²⁶)C(O)R²⁷, N(R²⁶)C(O)OR²⁷, N(R²⁶)C(O)N(R²⁶)(R²⁷), N(R²⁶)S(═O)₂R²⁶, CN, and OR²⁶, wherein the alkyl, cycloalkyl, heterocyclyl, aryl, and aryloxy are optionally substituted with from one to three substituents selected from alkyl, cycloalkyl, alkoxy, hydroxyl, and halo;

R²⁶ and R²⁷ are independently selected from the group consisting of hydrogen, alkyl, cycloalkyl, heterocyclyl, aryl, and heteroaryl, wherein the alkyl, cycloalkyl, heterocyclyl, aryl, and heteroaryl are optionally substituted with from one to three substituents selected from halo, alkyl, monoalkylamino, dialkylamino, alkyl amide, aryl amide, heteroaryl amide, CN, lower alkoxy, CF₃, aryl, and heteroaryl; or

R²⁶ and R²⁷ when taken together with the nitrogen to which they are attached form a heterocycle;

R²² is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, NO₂, haloalkyl, haloalkoxy, CN, OR²⁶, OC(O)R²⁶, OC(O)N(R²⁶)(R²⁷), SR²⁶, N(R²⁶)(R²⁷), S(═O)R²⁶, S(═O)₂R²⁶, S(═O)₂N(R²⁶)(R²⁷), S(═O)₂OR²⁶, N(R²⁶)C(O)R²⁷, N(R²⁶)C(O)OR²⁷, N(R²⁶)C(O)N(R²⁶)(R²⁷), C(O)R²⁶, C(O)OR²⁶, C(O)N(R²⁶)(R²⁷), and N(R²⁶)S(═O)₂R²⁷ and wherein the alkyl, alkoxy, cycloalkyl, aryl, heteroaryl and heterocyclyl are optionally substituted with one or more substituents selected from halo, oxo, NO₂, alkyl, haloalkyl, haloalkoxy, N(R²⁶)(R²⁷), C(O)R²⁶, C(O)OR²⁶, C(O)N(R²⁶)(R²⁷), CN, OR²⁶, cycloalkyl, aryl, heteroaryl and heterocyclyl; with the proviso that the heteroaryl or heterocyclyl moiety includes at least one ring nitrogen atom;

R²⁴ and R²⁵ are independently hydrogen, halo, cyano, alkyl, alkoxy, or cycloalkyl, wherein the alkyl, alkoxy, and cycloalkyl are optionally substituted by halo or cycloalkyl;

X²¹ and X²⁵ are independently C(R²³) or N, wherein each R²³ is independently hydrogen, halo, alkyl, alkoxy or cycloalkyl, wherein the alkyl and cycloalkyl are optionally substituted with from one to five substituents selected from halo, oxo, CF₃, OCF₃, N(R²⁶)(R²⁷), C(O)R²⁶, C(O)OR²⁷, C(O)N(R²⁶)(R²⁷), CN, and OR²⁶; and

X²², X²³ and X²⁴ are independently C(R²³), N, O, or S; with the proviso that at least one of X²², X²³, and X²⁴ is C(R²³); and only one of X²², X²³, and X²⁴ is O or S;

or a pharmaceutically acceptable salt, isomer, or a mixture thereof.

In some embodiment, the ASK1 inhibitor is the compound having the structure of formula (II), wherein:

R²¹ is C₁₋₆ alkyl, C₂₋₆ alkenyl, C₂₋₆ alkynyl, C₃₋₆ cycloalkyl, aryl, heteroaryl, or heterocyclyl, wherein the C₁₋₆ alkyl, C₂₋₆ alkenyl, C₂₋₆ alkynyl, C₃₋₆ cycloalkyl, aryl, heteroaryl, and heterocyclyl are optionally substituted with from one to four substituents selected from the group consisting of halo, hydroxyl, oxo, alkyl, cycloalkyl, heterocyclyl, aryl, aryloxy, NO₂, R²⁶, C(O)R²⁶, OC(O)R²⁶C(O)OR²⁶, C(O)N(R²⁶)(R²⁷), OC(O)N(R²⁶)(R²⁷), SR²⁶, S(═O)R²⁶, S(═O)₂R²⁶, S(═O)₂N(R²⁶)(R²⁷), S(═O)₂OR²⁶, N(R²⁶)(R²⁷), N(R²⁶)C(O)R²⁷, N(R²⁶)C(O)OR²⁷, N(R²⁶)C(O)N(R²⁶)(R²⁷), N(R²⁶)S(═O)₂R²⁶, CN, and OR²⁶, wherein the alkyl, cycloalkyl, heterocyclyl, aryl, and aryloxy are optionally substituted with from one to three substituents selected from alkyl, cycloalkyl, alkoxy, hydroxyl, and halo;

R²⁶ and R²⁷ are independently selected from the group consisting of hydrogen, alkyl, cycloalkyl, heterocyclyl, aryl, and heteroaryl, wherein the alkyl, cycloalkyl, heterocyclyl, aryl, and heteroaryl are optionally substituted with from one to three substituents selected from halo, alkyl, monoalkylamino, dialkylamino, alkyl amide, aryl amide, heteroaryl amide, CN, lower alkoxy, CF₃, aryl, and heteroaryl; or

R²⁶ and R²⁷ when taken together with the nitrogen to which they are attached form a heterocycle;

R²² is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, NO₂, haloalkyl, haloalkoxy, CN, OR²⁶, OC(O)R²⁶, OC(O)N(R²⁶)(R²⁷), SR²⁶, N(R²⁶)(R²⁷), S(═O)R²⁶, S(═O)₂R²⁶, S(═O)₂N(R²⁶)(R²⁷), S(═O)₂OR²⁶, N(R²⁶)C(O)R²⁷, N(R²⁶)C(O)OR²⁷, N(R²⁶)C(O)N(R²⁶)(R²⁷), C(O)R²⁶, C(O)OR²⁶, C(O)N(R²⁶)(R²⁷), and N(R²⁶)S(═O)₂R⁷, and wherein the alkyl, alkoxy, cycloalkyl, aryl, heteroaryl and heterocyclyl are optionally substituted with one or more substituents selected from halo, oxo, NO₂, alkyl, haloalkyl, haloalkoxy, N(R²⁶)(R²⁷), C(O)R²⁶, C(O)OR²⁶, C(O)N(R²⁶)(R²⁷), CN, OR²⁶, cycloalkyl, aryl, heteroaryl and heterocyclyl; with the proviso that the heteroaryl or heterocyclyl moiety includes at least one ring nitrogen atom;

R²⁴ and R²⁵ are independently hydrogen, halo, cyano, C₁₋₆ alkyl, C₁₋₆ alkoxy, or C₁₋₆ cycloalkyl, wherein the alkyl, alkoxy, and cycloalkyl are optionally substituted by halo or C₃₋₈ cycloalkyl;

X²¹ and X²⁵ are independently C(R²³) or N, wherein each R²³ is independently hydrogen, halo, C₁₋₆ alkyl, C₁₋₆ alkoxy or C₃₋₈ cycloalkyl, wherein the alkyl and cycloalkyl are optionally substituted with from one to five substituents selected from halo, oxo, CF₃, OCF₃, N(R²⁶)(R²⁷), C(O)R²⁶, C(O)OR²⁷, C(O)N(R²⁶)(R²⁷), CN, and OR²⁶; and

X²², X²³ and X²⁴ are independently C(R²³), N, O, or S; with the proviso that at least one of X²², X²³, and X²⁴ is C(R²³); and only one of X²², X²³, and X²⁴ is O or S;

or a pharmaceutically acceptable salt, isomer, or a mixture thereof.

Exemplary compounds of Formula (II) for use in the methods and pharmaceutical compositions described herein can be found in U.S. Patent Application Publication No. 2012/0004267, which is incorporated herein by reference in its entirety.

In certain embodiments, the ASK1 inhibitor is a compound of formula (III):

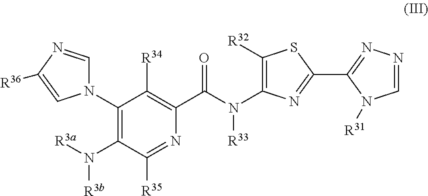

wherein:

R³¹ is alkyl or cycloalkyl, wherein the alkyl or cycloalkyl is optionally substituted with one to three halogen atoms;

R³² is hydrogen or alkyl wherein the alkyl is optionally substituted with halo.

R³³ is hydrogen or alkyl;

R³⁴ is hydrogen or alkyl;

R³⁵ is hydrogen, alkyl, OR^{3a} or —NHR^{3a};

R³⁶ is hydrogen, alkyl, haloalkyl, or C₃-C₆ cycloalkyl wherein the cycloalkyl is optionally substituted with alkyl, haloalkyl, or 1 or 2 halogen atoms;

R^{3a} and R^{3b} are independently hydrogen, alkyl or R^{3a} and R^{3b} combine with the nitrogen atom to which they are attached to form a four to six member heterocyclic ring optionally containing an oxygen or a nitrogen atom in the ring;

or a pharmaceutically acceptable salt, isomer, or mixture thereof.

In certain embodiment, the ASK1 inhibitor is a compound having the structure of formula (III), wherein:

R³¹ is C₁-C₃ alkyl or C₃-C₆ cycloalkyl, wherein the alkyl or cycloalkyl is optionally substituted with one to three halogen atoms;

R³² is hydrogen or C₁-C₆ alkyl wherein the alkyl is optionally substituted with halo.

R³³ is hydrogen or C₁-C₃ alkyl;

R³⁴ is hydrogen or C₁-C₃ alkyl;

R³⁵ is hydrogen, C₁-C₃ alkyl, OR^{3a} or —NHR^{3a};

R³⁶ is hydrogen, C₁-C₃ alkyl, C₁-C₃ haloalkyl, or C₃-C₆ cycloalkyl wherein the cycloalkyl is optionally substituted with C₁-C₃ alkyl, C₁-C₃ haloalkyl, or 1 or 2 halogen atoms;

R^{3a} and R^{3b} are independently hydrogen, C₁-C₃ alkyl or R^{3a} and R^{3b} combine with the nitrogen atom to which they are attached to form a four to six member heterocyclic ring optionally containing an oxygen or a nitrogen atom in the ring;

or a pharmaceutically acceptable salt, isomer, or mixture thereof.

Exemplary compounds of Formula (III) for use in the methods and pharmaceutical compositions described herein can be found in U.S. Patent Application Publication No. 2014/0179663, which is incorporated herein by reference in its entirety.

In some embodiments, the ASK 1 inhibitor are the compounds described in U.S. Patent Application Publication Nos. 2007/0276050, 2011/0009410, 2013/0197037, 2013/0197037, and 2014/0179663, 2014/0038957, 2014/0018370, 2009/0318425, 2011/0077235, 2012/0316194, U.S. Pat. No. 8,263,595, U.S. Provisional Patent Application No. 61/918,784, and PCT Patent Application Publication No. 2011/041293; all of which are incorporated herein by reference in their entirety. In certain embodiments, the ASK1 inhibitor is:

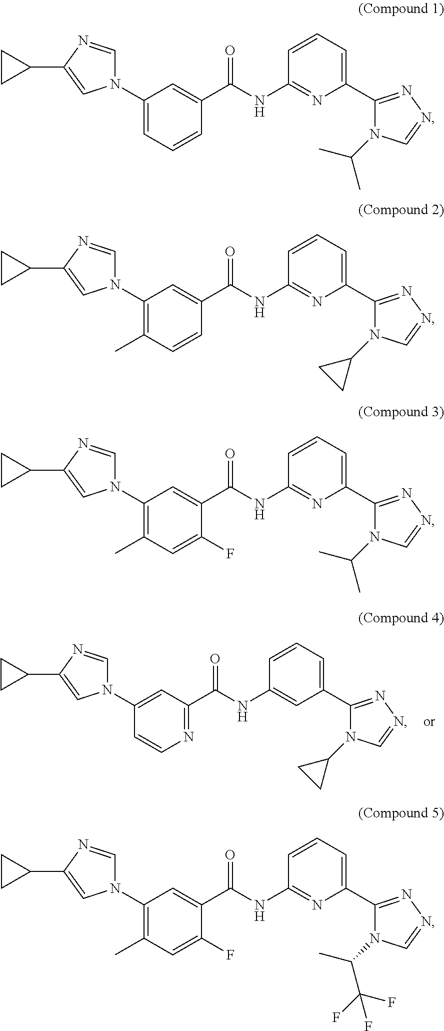 [SMILES: CC(C)n1cnnc1-c1cccc(NC(=O)c2cccc(-n3cnc(C4CC4)c3)c2)n1 ; Cc1ccc(C(=O)Nc2cccc(-c3nncn3C3CC3)n2)cc1-n1cnc(C2CC2)c1 ; Cc1cc(F)c(C(=O)Nc2cccc(-c3nncn3C(C)C)n2)cc1-n1cnc(C2CC2)c1 ; O=C(Nc1cccc(-c2nncn2C2CC2)c1)c1cc(-n2cnc(C3CC3)c2)ccn1 ; Cc1cc(F)c(C(=O)Nc2cccc(-c3nncn3[C@@H](C)C(F)(F)F)n2)cc1-n1cnc(C2CC2)c1]

or a pharmaceutically acceptable salt, isomer, or a mixture thereof. Compounds 1, 2, 3, 4, and 5 may be synthesized and characterized using the commonly used methods or those described in U.S. Patent Application Publication Nos. 2011/0009410 and 2013/0197037. In one embodiment, the ASK1 inhibitor is Compound 1 or a pharmaceutically acceptable salt thereof. In some embodiment, the ASK1 inhibitor is Compound 2 or a pharmaceutically acceptable salt thereof. In further embodiment, the ASK1 inhibitor is Compound 3 or a pharmaceutically acceptable salt thereof. In some further embodiment, the ASK1 inhibitor is Compound 4 or a pharmaceutically acceptable salt thereof. In certain further embodiment, the ASK1 inhibitor is Compound 5 or a pharmaceutically acceptable salt thereof.

The compounds of the present application may be represented by structures or chemical names. Also, the compounds may be named using the nomenclature systems and symbols that are commonly recognized in the art of chemistry including; for example, ChemBioDraw Ultra 12.0, Chemical Abstract Service (CAS), and International Union of Pure and Applied Chemistry (IUPAC). By way of example, Compound 3 may also be referred to as 5-(4-cyclopropyl-1H-imidazol-1-yl)-N-(6-(4-isopropyl-4H-1,2,4-triazol-3-yl)pyridin-2-yl)-2-fluoro-4-methylbenzamide, 5-(4-cyclopropylimidazol-1-yl)-2-fluoro-4-methyl-N-[6-(4-propan-2-yl-1,2,4-triazol-3-yl)pyridin-2-yl]benzamide, or 5-(4-cyclopropyl-1H-imidazol-1-yl)-2-fluoro-N-(6-(4-isopropyl-4H-1,2,4-triazol-3-yl)pyridin-2-yl)-4-methylbenzamide. Unless stated otherwise, the compounds described herein are named using ChemBioDraw Ultra 12.0; accordingly, Compound 1 may be referred to as 3-(4-cyclopropyl-1H-imidazol-1-yl)-N-(6-(4-isopropyl-4H-1,2,4-triazol-3-yl)pyridin-2-yl)benzamide, Compound 2 may be referred to as 3-(4-cyclopropyl-1H-imidazol-1-yl)-N-(6-(4-cyclopropyl-4H-1,2,4-triazol-3-yl)pyridin-2-yl)-4-methylbenzamide, Compound 3 may be referred to as 5-(4-cyclopropyl-1H-imidazol-1-yl)-2-fluoro-N-(6-(4-isopropyl-4H-1,2,4-triazol-3-yl)pyridin-2-yl)-4-methylbenzamide, Compound 4 may be referred to as 4-(4-cyclopropyl-1H-imidazol-1-yl)-N-(3-(4-cyclopropyl-4H-1,2,4-triazol-3-yl)phenyl)picolinamide, and Compound 5 may be referred to as (S)-5-(4-cyclopropyl-1H-imidazol-1-yl)-2-fluoro-4-methyl-N-(6-(4-(1,1,1-trifluoropropan-2-yl)-4H-1,2,4-triazol-3-yl)pyridin-2-yl)benzamide.

The present application provides pharmaceutically acceptable salts, hydrates, solvates, isomers, tautomers, stereoisomers, enantiomers, racemates, polymorphs, prodrugs, or a mixture thereof, of the compounds described herein. In addition, the present application provides the compounds that are labeled with or have at least one radioactive or non-radioactive isotope incorporated. By way of example, the compound in which from 1 to n hydrogen atoms attached to a carbon atom may be replaced by a deuterium atom or D, in which n is the number of hydrogen atoms in the molecule. It is known that the deuterium atom is a non-radioactive isotope of the hydrogen atom. Such compounds may increase resistance to metabolism, and thus may be useful for increasing the half-life of the compounds of any of the formulae described herein or pharmaceutically acceptable salts, isomers, prodrugs, or solvates thereof, when administered to a mammal (see, e.g., Trends Pharmacol. Sci. 1984; 5(12):524-527). Such compounds may be synthesized by means well known in the art, for example by employing starting materials in which one or more hydrogen atoms have been replaced by deuterium. Also, the compound in which 1 to n carbon atoms may be replaced by ¹⁴C atoms. Other suitable isotopes include and are not limited to ¹¹C, ¹²C, ¹³C, ¹⁵C, ¹³N, ¹⁵O, and ¹⁸F. The labeled compounds are useful in characterizing the properties of the compounds (e.g. biodistribution in vivo) or for diagnosing purposes and may be synthesized by means well known in the art.

The terms “a compound of the present application,” “a compound described herein,” “a compound of any of the formulae described herein,” or variant thereof refer to a compound having the structure of any of the formulae (I), (IA), (II), or (III).

“Isomers” refers to compounds that have the same molecular formula. As used herein, the term isomers include double bond isomers, racemates, stereoisomers, enantiomers, diastereomers, and atropisomers. Single isomers, such as enantiomers or diastereomers, can be obtained by asymmetric synthesis or by resolution of a mixture of isomers. Resolution of a mixture of isomers (e.g. racemates) maybe accomplished, for example, by conventional methods such as crystallization in the presence of a resolving agent, or chromatography, using, for example a chiral high pressure liquid chromatography (HPLC) column. “Double bond isomers” refer to Z- and E-forms (or cis- and trans-forms) of the compounds with carbon-carbon double bonds.

“Racemates” refers to a mixture of enantiomers.

“Stereoisomers” or “stereoisomeric forms” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomers” or “tautomeric formers” refer to alternate forms of a compound that differ in the position of a proton, such as enol-keto and imine-enamine tautomers, or heteroaryls such as pyrazoles, imidazoles, benzimidazoles, triazoles, and tetrazoles.

A “solvate” is formed by the interaction of a solvent and a compound. Solvates of salts of the compounds of any of the formulae described herein are also provided. Hydrates of the compounds of any of the formulae are also provided.

A “prodrug” is defined in the pharmaceutical field as a biologically inactive derivative of a drug that upon administration to the human body is converted to the biologically active parent drug according to some chemical or enzymatic pathway.

The application further provides compositions comprising the compounds described herein or a pharmaceutically acceptable salt, isomer, prodrug, or solvate thereof. The composition may include racemic mixtures, mixtures containing an enantiomeric excess of one enantiomer or single diastereomers or diastereomeric mixtures. All such isomeric forms of these compounds are expressly included herein, the same as if each and every isomeric form were specifically and individually listed. The application also provides a composition containing a mixture of enantiomers of the compound or a pharmaceutically acceptable salt thereof. In one embodiment, the mixture is a racemic mixture, the mixture containing the (S)-enantiomer of a compound in excess of over the corresponding the (R)-enantiomer of the compound, or a mixture containing less than or about 40%, 35%, 30%, 25%, 20%, 15%, 10%, 5%, 1%, 0.05%, or 0.01% of the (R)-enantiomer. In other embodiments, the composition containing the (S)-enantiomer of a compound or a pharmaceutically acceptable salt thereof, predominates over its corresponding (R)-enantiomer by a molar ratio of at least or about 9:1, at least or about 19:1, at least or about 40:1, at least or about 80:1, at least or about 160:1, or at least or about 320:1, or containing the (S)-enantiomer of the compound and is substantially free of its corresponding (R)-enantiomer.

In certain embodiments, provided herein are also polymorphs, such as crystalline and amorphous forms, of the compounds described herein. In some embodiments, provided are also chelates, non-covalent complexes, and mixtures thereof, of the compounds of the formula described herein or pharmaceutically acceptable salts, prodrugs, or solvates thereof. A “chelate” is formed by the coordination of a compound to a metal ion at two (or more) points. A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding).

Pulmonary Hypertension

Pulmonary hypertension (PH) is a pulmonary vascular disease that is characterized by an increase in mean pulmonary arterial pressure (PAP) ≧25 mmHg at rest as determined by right heart catheterization (RHC). Pulmonary hypertension may be found in multiple clinical conditions and has been classified into different clinical groups (J. Am. Coll. Cardiol. 2013; 62(25 Suppl):D34-41). Group 1 is pulmonary arterial hypertension (PAH) which is further divided into five subgroups based on disease pathology: idiopathic PAH, in which the cause of the disease is unknown (1.1); heritable PAH (previously referred to as familial PAH) (1.2), which is inherited or is due to specific gene mutations including bone morphogenetic protein receptor type 2 (BMPR2) (1.2.1), ALK-1 (active receptor-like kinase 1 gene) endoglin (with or without hereditary haemorrhagic telangiectasia) (1.2.2), and unknown (1.2.3); drugs and toxins induced PAH (1.3) including causes by diet drugs, pulmonary embolism, or the like; associated with PAH (APAH) (1.4) which is caused by other conditions including connective tissue diseases (1.4.1), HIV infection (1.4.2), portal hypertension (1.4.3), congenital heart disease (1.4.4), and schistosomiasis (1.4.5). The diagnosis of PAH requires the exclusion of all other groups (Eur. Respir. J. 2009; 34:1219-1263).

Group 1′ includes pulmonary veno-occlusive disease and/or pulmonary capillary haemangiomatosis, and Group 1″ includes persistent pulmonary hypertension of the newborn (PPHN). In addition, Groups 2-5 are pulmonary hypertension due to various other causes. Group 2 is pulmonary hypertension due to left heart disease and is further divided into systolic dysfunction (2.1), diastolic dysfunction (2.2), valvular disease (2.3), and congenital/acquired left heart inflow/outflow tract obstruction and congenital cardiomyopathies (2.4). Group 3 is pulmonary hypertension due to lung diseases and/or hypoxia and may be caused by chronic obstructive pulmonary disease (3.1), interstitial lung disease (3.2), other pulmonary diseases with mixed restrictive and obstructive pattern (3.3), sleep-disordered breathing (3.4), alveolar hypoventilation disorders (3.5), chronic exposure to high altitude (3.6), and developmental abnormalities (3.7). Group 4 is chronic thromboembolic pulmonary hypertension. Group 5 is pulmonary hypertension with unclear and/or multifactorial mechanisms that are associated with hematological disorders: chronic hemolytic anemia, myeloproliferative disorders, and splenectomy (5.1); systemic disorders including sarcoidosis, pulmonary Langerhans cell histiocytosis, lymphangioleiomyomatosis, neurofibromatosis, and vasculitis (5.2); metabolic disorders: glycogen storage disease, Gaucher disease, and thyroid disorders (5.3); or others such as tumoural obstruction, fibrosing mediastinitis, chronic renal failure, and segmental P1H.

Pulmonary arterial hypertension (PAH) or Group I pulmonary hypertension (PH) is characterized by continuous high blood pressure in the pulmonary arteries. For example, in a healthy individual or human, mean PAP is ≦15 mmHg when resting. However, in PAH patients, mean PAP is usually ≧25 mmHg. The pulmonary arteries are the blood vessels that carry oxygen-poor blood from the right ventricle of the heart to the small arteries in the lungs, providing blood with fresh oxygen. Once this oxygenated blood leaves the lungs, it goes back to the heart to be pumped out to all parts of the body, delivering oxygen and nutrients to tissues and organs. When the pressure is high in the pulmonary arteries (for example, due to pulmonary hypertension), the right side of the heart has to work much harder to get blood into the lungs, causing shortness of breath, fatigue, chest pain, heart palpitations, and/or fainting, which may occur with or without exertion, and leading to right ventricle dysfunction and/or failure.

In PAH, three types of changes may occur in the pulmonary arteries: (i) the smooth muscle layer within the walls of the arteries may persistently constrict which makes the inside of the arteries narrower; (ii) the walls of the pulmonary arteries may thicken as the amount of muscle increases and the scar tissue may form in the walls of arteries, causing the arteries become increasingly narrower; and (iii) tiny blood clots may form within the smaller arteries, causing blockages. As a result of these changes, there is less room for the blood to flow through these narrower arteries. The narrowing or complete blockage of the pulmonary arteries may cause the right ventricle (RV) of the heart to work harder (i.e. RV pressure overload) to pump blood through the lungs. With pressure overload over time, the RV myocardium hypertrophies, and then dilates, causing the heart muscle to weaken to such an extent that the heart loses its ability to pump enough blood through the body. This is commonly referred to as right heart failure which is the most common cause of death in people with PAH.

Symptoms of PAH result from a reduction in the amount oxygen delivered to the body due to narrowed or restricted pulmonary vasculature and increased stress on the heart. Symptoms may not be initially obvious but progress to become more limiting over time. The common symptoms of PAH include but are not limited to breathlessness or shortness of breath (dyspnea), fatigue (feeling tired all the time), dizziness (especially when climbing stairs or when standing up), fainting (syncope), swollen ankles and legs (edema), or chest pain (angina) (especially during physical activity). When left untreated, the patients will suffer heart failure and death. Drugs or therapeutics that have been investigated for the treatment of PAH include the following classes: calcium channel blockers, prostanoids, endothelin receptor antagonists, phosphodiesterase type 5 inhibitors, cGMP activators, vasoactive intestinal peptides, nonprostanoid prostacyclin receptor agonists, tyrosine kinase inhibitors (platelet-derived growth factor receptor inhibitors), and serotonin antagonists. Although some treatments within these classes have been approved, the PAH patients with PAH still face a poor prognosis of 68% survival at 3 years. This is likely due to the underlying, progressive maladaptive remodeling processes (e.g., cellular hyperplasia, hypertrophy, inflammation, migration, and extracellular matrix deposition) in the pulmonary vasculature, resulting in a progressive increase in PVR and ultimately RV dysfunction and failure. Currently there is no cure for PAH, and it remains a chronic disease.

Previous studies have shown that the expression of p38, a kinase downstream of ASK1, is increased in lungs from idiopathic PAH patients (Thorax 2012; 67:A19-A20). In addition, the proliferation of human pulmonary artery smooth muscle cells in response to bone morphogenetic protein 4 (BMP-4) is dependent on p38 kinase signaling (Am. J. Respir. Cell Mol. Biol. 2007; 37(5):598-605). Studies also show that p38 inhibition may attenuate the hypoxia-induced proliferation of human pulmonary artery cells (Pul. Pharm. & Thera. 2007; 20(6):718-25). The downstream substrates of ASK1, including p38 and c-Jun N-terminal kinase (JNK), mediate diverse cellular responses by phosphorylating both cytosolic substrates and nuclear transcription factors (EMBO reports 2001; 2(3):222-8). Studies have shown that the activation of ASK1 pathway induces the expression of inflammatory cytokines (e.g., IL-1β, IL-2, and IL-6), chemokines (e.g., monocyte chemotactic protein 1 (MCP-1), chemokine ligand 1 (CXCL1), and chemokine ligand 2 (CXCL2)), and matrix remodeling genes (e.g. TGF-β, TIMP, and PAI-1) (Nat. Immun. 2005; 6(6):587-92).

In patients with PAH, oxidative stress is increased and antioxidant capacity is reduced (Eur. Respir. J. 2009; 34(1):276; Am. J. Respir. Crit. Care Med. 2004; 169(6):764-9). In addition, elevated levels of the circulating oxidative stress biomarkers 8-isoprostane F2α, uric acid, and asymmetric dimethylarginine are seen in patients with PAH and have been associated with poor outcomes (Arterioscler. Thromb. Vasc. Biol. 2005; 25(7):1414-8).

The present application described results showing that ASK1 is a potential therapeutic target. Without being bound to any theories, the ASK1 signaling pathway may be involved in oxidative stress-induced injury in inducing or causing PAH. ASK1 has been shown to be expressed in various tissues and bound and repressed by thiol-containing antioxidant proteins, including thioredoxins in the cytosol and mitochondria (Mol. Cell Biol. 2007; 27(23):8152-63). In elevated or increased oxidative stress and/or ROS, thioredoxin undergoes oxidation and dissociation from ASK1; leading to trans-autophosphorylation of ASK1 homodimers at Threonine 845 (ASK-T₈₄₅) within the activation loop (J. Cell. Phys. 2002; 191(1):95-104). Phospho-ASK1-T₈₄₅ phosphorylates Mitogen-Activated Protein Kinases (MAPKK) 3, 4, 6, and 7, which in turn phosphorylate and activate the Mitogen-Activated Protein Kinase (MAPK) p38 and c-Jun N-terminal kinase (JNK) (Annu. Rev. Pharmacol. Toxicol. 2008; 48:199-2).

As described in the present application, ASK1 inhibitor, such as Compound 3, prevented the activation of ASK1, reduced the phosphorylation of p38 MAPK. Additionally, ASK1 inhibitor, such as Compound 4, dose-dependently decreased pulmonary arterial pressure and RV hypertrophy in an in vivo model of PH. The results described herein indicate that ASK1 inhibition reduced hallmarks of pulmonary vascular disease including and not limited to reduced PVR, improved pulmonary pressure, decreased pulmonary vascular remodeling, improved vascular function, decreased maladaptive RV hypertrophy, and improved RV function. This suggests that inhibition of ASK1 signaling may slow, prevent, and/or reverse pathological changes associated with PH.

The present application provides a method of treating and/or preventing pulmonary vascular disease by administering a therapeutically effective amount of ASK1 inhibitor. In one embodiment, the pulmonary vascular disease is pulmonary hypertension. In other embodiment, the pulmonary vascular disease is pulmonary hypertension Group 1, 1′, 1″, 2, 3, 4, or 5. In some other embodiment, the pulmonary vascular disease is pulmonary arterial hypertension. In additional embodiment, the ASK1 inhibitor is a compound having the structure of formulae (I), (IA), (II), (III), or a pharmaceutically acceptable salt, isomer, or a mixture thereof. In certain embodiment, the ASK1 inhibitor is selected from Compound 1, 2, 3, 4, 5, or a pharmaceutically acceptable salt thereof. In certain other embodiment, the method of treating and/or preventing pulmonary arterial hypertension comprises administering Compound 3 or a pharmaceutically acceptable salt thereof.

As described in U.S. Patent Application Publication No. 2013/0197037, Compound 3 is a potent and selective inhibitor of ASK1. Without being bound to any hypothesis, ASK1 inhibitor, such as the compounds of formula (I), (IA), (II), and (III), may provide therapeutic effects via multiple mechanisms; for example, reducing or inhibiting ASK1 signaling, proliferation, inflammation, oxidative stress, and/or RV maladaptive remodeling. The therapeutics based on the ASK1 inhibitor (e.g. Compounds 3 and 4) may improve pulmonary arterial (or cardiopulmonary) hemodynamics, functional capacity, symptoms, and/or RV function; thus reducing morbidity or mortality.

Therapeutic Uses of the Compounds

The compounds of the formulae described herein or a pharmaceutically acceptable salt, isomer, prodrug, or solvate thereof may be used for the treatment of pulmonary hypertension including but not limited to pulmonary arterial hypertension. In addition, the application provides the compounds for use in therapy. Also, provided herein are methods for inhibiting ASK1. In one embodiment, provided are methods for inhibiting ASK1 activity using the compound described herein or a pharmaceutically acceptable salt, isomer, prodrug, or solvate thereof. In other embodiment, provided are methods for inhibiting ASK1 signaling using the compound or a pharmaceutically acceptable salt, isomer, prodrug, or solvate thereof. In other embodiment, provided are methods for inhibiting ASK1, p38, and/or JNK activities using the compound or a pharmaceutically acceptable salt, isomer, prodrug, or solvate thereof. The application further provides methods for use in such methods. Additionally, the compounds may be used to inhibit ASK1 activity or signaling therapeutically or prophylactically.

The compounds according to the present application may be used in combination with one or more additional therapeutic agents. The therapeutic agents may be in the forms of compounds, antibodies, polypeptides, or polynucleotides. The therapeutic agent includes, but is not limited to, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. In one embodiment, the application provides a product comprising a compound described herein and a therapeutic agent as a combined preparation for simultaneous, separate or sequential use in therapy, e.g. a method of treating pulmonary hypertension including but not limited to pulmonary arterial hypertension.

In some embodiments, the therapeutic agents may be those that inhibit or modulate the activities of Bruton's tyrosine kinase, spleen tyrosine kinase, apoptosis signal-regulating kinase, Janus kinase, lysyl oxidase, lysyl oxidase-like proteins, matrix metallopeptidase, bromodomain-containing protein, adenosine A2B receptor, isocitrate dehydrogenase, serine/threonine kinase TPL2, discoidin domain receptor, serine/threonine-protein kinases, IKK, MEK, EGFR, histone deacetylase, protein kinase C, or any combination thereof. In certain embodiments, the therapeutic agents may be vasodilators, angiotensin-converting-enzyme (ACE) inhibitors, beta blockers, calcium channel blockers, prostanoids, endothelin receptor antagonists, phosphodiesterase type 5 inhibitors, cGMP activators, vasoactive intestinal peptides, nonprostanoid prostacyclin receptor agonists, prostacyclin receptor agonists, tyrosine kinase inhibitors (platelet-derived growth factor receptor inhibitors), serotonin antagonists, or any combination thereof. In certain other embodiments, the therapeutic agents may be anticoagulants, diuretics, oxygen, or digoxin. In additional embodiment, the therapeutic agent is selected from the group consisting of diuretics, beta blockers, ACE inhibitors, prostaglandins (prostacyclin derivatives, epoprostenol (Flolan®), treprostinil (Remodulin®), treprostinil (Tyvaso®), treprostinil (Orenitram®), iloprost (Ventavis®)), endothelin receptor antagonists (ambrisentan (Letairis®), bosentan (Tracleer®), macitentan(Opsumit®)), phosphodiesterase type 5 (PDE-5) inhibitors (sildenafil (Revatio®), tadalafil (Adcirca®)), soluble guanylate cyclase activators (riociguat (Adempas®)), prostacyclin receptor agonists (selexipag), or a combination thereof. In one embodiment, the ASK1 inhibitor may be used in combination with one, two, or three therapeutic agents described above.

Provided herein is a method of treating and/or preventing pulmonary vascular disease in a patient in need thereof, comprising administering to the patient a therapeutically effective amount of an ASK1 inhibitor. In certain embodiments, the pulmonary vascular disease is a pulmonary arterial hypertension (PAH). In some embodiments, the patient is diagnosed with Group 1, 1′, 1″, 2, 3, 4, or 5 pulmonary hypertension.

Provided herein is a method of treating and/or preventing right ventricle dysfunction in a patient in need thereof, comprising administering to the patient a therapeutically effective amount of an ASK1 inhibitor.

Also provided herein is a method of treating, preventing, and/or reversing the narrowing or restricting of pulmonary arteries in a patient in need thereof, comprising administering to the patient a therapeutically effective amount of an ASK1 inhibitor.

Additionally, provided herein is a method of reducing or normalizing high mean pulmonary arterial pressure (mPAP) and/or high pulmonary vascular resistance in a patient in need thereof, comprising administering to the patient a therapeutically effective amount of an ASK1 inhibitor. In one embodiment, the high mPAP ≧25 mmHg at rest may be reduced to levels within the normal range at rest by the methods described herein. In some embodiment, the high mPAP ≧25 mmHg at rest may be reduced to about 22 mmHg, 20 mmHg, 18 mmHg, 16 mmHg, or 14 mmHg at rest by the methods described herein. In certain embodiment, mPAP is determined by right heart catheterization (RHC).

Provided herein is a method of improving or reducing PAH symptoms in a patient in need thereof, comprising administering a therapeutically effective amount of ASK1 inhibitor. In some embodiments, PAH symptoms include and are not limited to breathlessness or shortness of breath (dyspnea), fatigue, dizziness, fainting (syncope), swollen ankles and legs (edema), chest pain, right heart failure and/or dysfunction. In certain embodiments, the improvement may be determined by a change from baseline in pulmonary vascular resistance (PVR), a change from baseline in cardiac index (CI) such as mean pulmonary artery pressure (mPAP), mean right atrial pressure (mRAP), mixed venous oxygen saturation (SvO₂), and right ventricular cardiac power, a change from baseline in clinical measures of symptoms and function, including but not limited to submaximal exercise (6-minute walk test (6MWT)), heart rate recovery (HRR) after the 6MWT, the Borg dyspnea index, WHO Functional Class, N-terminal pro-brain natriuretic peptide, and/or quality of life by the SF-36® Health Survey. In other embodiments, PVR is determined by right heart catheterization. In additional embodiments, cardiac function is determined by echocardiography or cardiac hemodynamic data.

Also provided herein is a method of improving pathological consequence or outcome associated with oxidative stress in a patient in need thereof comprising administering to the patient a therapeutically effective amount of ASK1 inhibitor.

Additionally provided herein is a method of reducing the remodeling of pulmonary vasculature or arteries in a patient in need thereof, comprising administering to the patient a therapeutically effective amount of an ASK1 inhibitor.

Further provided herein is a method of treating and/or preventing right ventricle failure or right ventricle dysfunction in a patient in need thereof comprising administering to the patient a therapeutically effective amount of an ASK1 inhibitor. In one embodiment, the right ventricle failure or dysfunction may be detected or monitored by cardiac imaging such as echocardiography and cardiac MRI.

Provided herein is a method of improving and/or reducing PVR, pulmonary pressure, pulmonary vascular remodeling, vascular function, maladaptive RV hypertrophy, and/or RV function in a patient in need thereof, comprising administering to the patient a therapeutically effective amount of an ASK1 inhibitor.

The present application provides a therapy or treatment to a patient in need, wherein the patient has or is suspected to have pulmonary vascular disease such as pulmonary hypertension or pulmonary arterial hypertension. In one embodiment, the patients experience one or more symptoms selected from breathlessness or shortness of breath (dyspnea), fatigue, dizziness, fainting (syncope), swollen ankles and legs (edema), or chest pain (e.g. angina). The patients may be at various clinical or treatment stages, including patients who have not received any prior treatment to pulmonary hypertension or pulmonary arterial hypertension, patient who have received prior therapies or drugs for pulmonary hypertension or pulmonary arterial hypertension and remains symptomatic, and patients who currently receive other therapies or drugs for pulmonary hypertension or pulmonary arterial hypertension. For example, the patient may have received the therapeutics of the present application (e.g. the ASK 1 inhibitor or a pharmaceutical composition thereof) and other PAH drugs concurrently.

In any of the foregoing, the treatment, prevention, reduction, reversion, and/or improvement by the method described herein may be determined by a change from baseline in pulmonary vascular resistance (PVR), a change from baseline in cardiac index (CI) such as mean pulmonary artery pressure (mPAP), mean right atrial pressure (mRAP), mixed venous oxygen saturation (SvO₂), and right ventricular cardiac power, a change from baseline in clinical measures of symptoms and function, including but not limited to submaximal exercise (6-minute walk test (6MWT)), heart rate recovery (HRR) after the 6MWT, the Borg dyspnea index, WHO Functional Class, N-terminal pro-brain natriuretic peptide, an/or quality of life by the SF-36® Health Survey. In other embodiments, PVR is determined by right heart catheterization. In additional embodiments, cardiac function is determined by echocardiography or cardiac hemodynamic data. The baseline refers to a value, number, or reading that is determined or measured from the subject prior to any treatment. By way of example, the baseline is a value, number, or reading from a patient prior to being treated with the methods described herein, from a healthy individual, from a group of subjects, or from suitable guidelines. In one embodiment, the baseline is a value, number, or reading from a patient prior to being treated with the methods described herein. The baseline value or number may be determined or measured by any suitable methods.

As used herein, the terms “right ventricle (RV) dysfunction,” “right ventricular dysfunction,” “right heart failure,” or variants thereof refer to the failure of right ventricle or right heart is unable to carry out the normal function (e.g. pumping blood out of the heart into the lungs to be replenished with oxygen, and/or maintaining sufficient blood flow to meet the needs of the body). RV dysfunction may be determined or detected by cardiac imaging including echocardiography and cardiac MRI which characterizes structural changes (myocardial hypertrophy followed by progressive contractile dysfunction and chamber dilation) and/or functional changes (reduced fractional shortening, increased filling pressures, reduced right ventricular ejection fraction and decreased cardiac output). Other commonly used methods may also be used to determine or detect RV dysfunction. Also, ‘promoting” or “stimulating” refer to one or more factor that may cause or contribute to progressing of activity, disease, disorder, or condition. For example, promoting or contributing to PAH is used to describe one or more factor that may cause or contribute to progressing or developing of PAH.

Dosing and Administration

While it is possible for an active ingredient (i.e., the ASK1 inhibitor) to be administered alone, it may be preferable to present them as pharmaceutical formulations or pharmaceutical compositions as described below. The formulations, both for veterinary and for human use, of the disclosure comprise at least one of the active ingredients (i.e., the ASK1 inhibitor), together with one or more acceptable carriers therefor and optionally other therapeutic ingredients. The carrier(s) must be “acceptable” in the sense of being compatible with the other ingredients of the formulation and physiologically innocuous to the recipient thereof.

The active ingredients may be administered under fed conditions. The term “fed conditions” or variations thereof refer to the consumption or uptake of food, in either solid or liquid forms, or calories, in any suitable form, before or at the same time when the active ingredients are administered. For example, the active ingredients may be administered to the subject (e.g., a human) within minutes or hours of consuming calories (e.g., a meal). In some embodiments, the active ingredients may be administered to the subject (e.g., a human) within 5-10 minutes, about 30 minutes, or about 60 minutes of consuming calories.

The active ingredient (i.e. the ASK1 inhibitor described herein) may be administered to the subject (e.g. a patient having pulmonary hypertension) every day continuously for a treatment period of 16, 20, 24, 28, or 32 weeks. The treatment period may be repeated one, two, three, or four times or continued indefinitely. Also, the active ingredient may be administered to the subject for six months, eight months, ten months, twelve months, sixteen months, or eighteen months, two years, three years, four years, or for an indefinite period of time. In addition, the treatment period may be repeated after a treatment-free gap of one day, two days, three days, four days, five days, six days, one week, two weeks, three weeks, four weeks, one month, two months, or three months.

During the treatment period, the subjects or patients may be assessed or monitored at various time points, for example, week 2, week 4, week 6, week 8, week 10, week 12, week 14, week 16, week 18, week 20, week 22, week 24, week 26, week 28, week 30, week 32, week 34, and/or week 36. The subjects or patients may be assessed or monitored for various variables, including and not limited to a change from baseline in pulmonary vascular resistance (PVR), a change from baseline in cardiac index (CI) such as mean pulmonary artery pressure (mPAP), mean right atrial pressure (mRAP), mixed venous oxygen saturation (SvO₂), and right ventricular cardiac power, a change from baseline in clinical measures of symptoms and function, including but not limited to submaximal exercise (6-minute walk test (6MWT)), heart rate recovery (HRR) after the 6MWT, the Borg dyspnea index, WHO Functional Class, N-terminal pro-brain natriuretic peptide, and/or quality of life by the SF-36® Health Survey. Other variables suitable to determine or measure the pulmonary vascular function and/or right ventricular function may be used; for example, echocardiography which provides non-invasive measures of cardiac function and other cardiac hemodynamic data.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

The therapeutically effective amount of active ingredient (i.e., the ASK1 inhibitor) can be readily determined by a skilled clinician using conventional dose escalation studies. Typically, the active ingredient will be administered in a dose from about 0.01 milligrams (mg) to 2 grams (g), about 0.1 mg to 450 mg, about 0.5 mg to about 250 mg, about 0.5 mg to 100 mg, about 0.5 mg to 50 mg, about 0.5 mg to 40 mg, about 0.5 mg to 30 mg, about 0.5 mg to 20 mg, about 0.5 mg to 10 mg, about 0.5 mg to 5 mg, about 0.5 mg to 4 mg, about 0.5 mg to 3 mg, about 0.5 mg to 2 mg, about 0.5 mg to 1 mg, about 1 mg to 250 mg, about 1 mg to 100 mg, about 1 mg to 50 mg, about 1 mg to 40 mg, about 1 to 35 mg, about 1 mg to 30 mg, about 1 to 25 mg, about 1 mg to 20 mg, about 1 to 15 mg, about 1 mg to 10 mg, about 1 mg to 5 mg, about 1 mg to 4 mg, about 1 mg to 3 mg, or about 1 mg to 2 mg. In another embodiment, the dosage ranges from about 1 mg or 100 mg. In some other embodiment, the dosage ranges from about 1 mg to 30 mg. In certain other embodiment, the dosage ranges from about 1 mg to 20 mg. In one embodiment, the dosage is about 0.5, 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92. 94, 96, 98, or 100 mg. It is contemplated that the active ingredient may be administered once, twice, or three times a day. Also, the active ingredient may be administered once or twice a week, once every two weeks, once every three weeks, once every four weeks, once every five weeks, or once every six weeks. In other embodiment, the active ingredient (i.e. Compound 1) is administered once daily at the dose of 1, 2, 6, 10, 18, 20, 30, or 100 mg.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets, or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

In one embodiment, the ASK1 inhibitor is presented in a tablet form. In certain embodiment, the ASK1 inhibitor is the compound having the formula (I), a pharmaceutically acceptable salt, isomer, or a mixture thereof in a tablet form. In some embodiment, the ASK1 inhibitor is Compound 1 or a pharmaceutically acceptable salt thereof in a tablet form. In additional embodiment, Compound 1 is in a tablet at a dose unit of 1, 2, 6, 10, 18, and 100 milligrams (mg) and the tablets contain pharmaceutically acceptable excipients.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In certain embodiments, the ASK1 inhibitor is administered with food. In one embodiment, the patient is human.

When used in combination in the methods disclosed herein, the ASK1 inhibitor and one or more therapeutic agent may be administered together in a single pharmaceutical composition, or separately (either concurrently or sequentially) in more than one pharmaceutical composition. In certain embodiments, the ASK1 inhibitor and the therapeutic agent are administered together. In other embodiments, the ASK1 inhibitor and the therapeutic agent are administered separately. In some aspects, the ASK1 inhibitor is administered prior to the one or more therapeutic agent. In some aspects, the one or more therapeutic agent is administered prior to the ASK1 inhibitor. When administered separately, the ASK1 inhibitor and the therapeutic agent may be administered to the patient by the same or different routes of delivery. For example, both may be administered orally, or the ASK1 inhibitor is administered orally and the one or more therapeutic agent may be administered subcutaneously.

Pharmaceutical Compositions

The pharmaceutical compositions described herein provide for an effective amount of an ASK1 inhibitor, such as the compounds having the foregoing formulae, a pharmaceutically acceptable salt, isomer, or a mixture thereof. In some embodiments, a pharmaceutical composition provides an effective amount of the compound having the formula (I), a pharmaceutically acceptable salt, isomer, or a mixture thereof. In certain embodiments, the pharmaceutical composition provides an effective amount of Compound 1, Compound 2, Compound 3, Compound 4, and Compound 5, the pharmaceutically acceptable salt, isomer, or a mixture thereof. In other embodiments, a pharmaceutical composition provides an effective amount of Compound 1 or a pharmaceutically acceptable salt thereof.

When used for oral use for example, tablets, troches, lozenges, aqueous or oil suspensions, dispersible powders or granules, aerosol, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

In one embodiment, the pharmaceutical composition comprising the ASK1 inhibitor is in a tablet form. In certain embodiment, the pharmaceutical composition comprising the ASK1 inhibitor is in a tablet form, wherein the ASK1 inhibitor is the compound having the formula (I), a pharmaceutically acceptable salt, isomer, or a mixture thereof. In some embodiment, the pharmaceutical composition comprising Compound 1 or a pharmaceutically acceptable salt thereof is in a tablet form. In additional embodiment, the pharmaceutical composition comprising Compound 1 is in a tablet at a dose unit of 1, 2, 6, 10, 18, and 100 milligrams (mg) and the tablets contain at least one pharmaceutically acceptable excipient.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions may contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the present application may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the present application may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material (i.e., an ASK1 inhibitor) compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In embodiments where the ASK1 inhibitor is administered in combination with one or more therapeutic agent, the ASK1 inhibitor and the therapeutic agent may be administered together in a combination formulation or in separate pharmaceutical compositions, where each of ASK1 inhibitor and the therapeutic agent may be formulated in any suitable dosage form. In certain embodiments, the methods provided herein comprise administering separately a pharmaceutical composition comprising the ASK1 inhibitor and a pharmaceutically acceptable carrier or excipient and a pharmaceutical composition comprising the therapeutic agent and a pharmaceutically acceptable carrier or excipient. Combination formulations according to the present disclosure comprise the ASK1 inhibitor and one therapeutic agent together with one or more pharmaceutically acceptable carriers or excipients and optionally other therapeutic agents. Combination formulations containing the active ingredient (i.e. an ASK1 inhibitor and the therapeutic agent) may be in any form suitable for the intended method of administration.

### EXAMPLES

The following examples are provided to further aid in understanding the embodiments disclosed in the application, and presuppose an understanding of conventional methods well known to those persons having ordinary skill in the art to which the examples pertain. The materials and conditions described hereunder are intended to exemplify certain aspects of embodiments disclosed herein and should not be construed to limit the reasonable scope thereof. It is understood that the assays may produce results that vary and may be within 1 to 3-fold of the reported mean. All of the patents, applications, publications, and literatures are incorporated herein by reference in the entirety.

### Example 1

### Characterization of ASK1 Inhibitor in an Acute Model of Oxidative Stress in the Right Ventricle (RV)

In this study, the auranofin model of oxidative stress-induced ASK1 activation was used to determine the effects of ASK1 inhibitor in preventing or inhibiting oxidative stress-induced activation of the ASK1 pathway in the rat RV. ASK1 is normally bound and repressed by the thiol-containing antioxidant protein thioredoxin 1 (Trx1). Auranofin (2,3,4,6-tetra-O-acetyl-1-thio-β-d-glucopyranosato-S-(triethylphosphine) gold) is a known inhibitor of thioredoxin reductase, whose activity is essential to prevent oxidation of Trx1. It has been shown that auranofin treatment results in Trx1 oxidation, thus promoting ASK1 autophosphorylation and activation. When administered in vivo, auranofin results in ASK1-mediated phosphorylation of p38MAPK, which in turn promotes induction of cytokine/chemokine gene expression.

Sprague-Dawley rats (n=5 to 8 per group) were administered with a single oral dose (0.3, 1, 3, or 10 mg/kg) of Compound 3 or an equal volume of vehicle. Rats were then challenged with a single intraperitoneal (ip) injection of auranofin at 30 mg/kg to induce oxidative stress. The levels of phosphorylated p38 in RV lysates were evaluated by Western blot analysis and normalized to IP90.

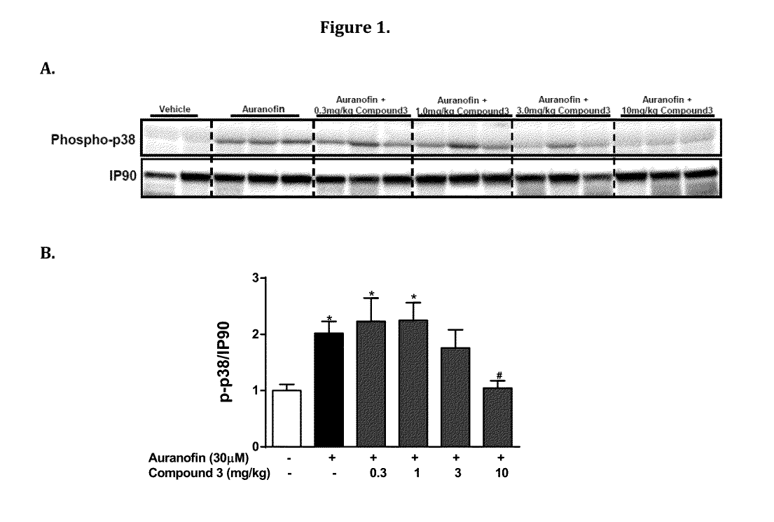

As shown in FIG. 1, the group treated with auranofin exhibited an increase in p38 phosphorylation in the RV (2.0±0.2) (mean±standard error of mean (SEM)) compared with the group treated with vehicle (1.0±0.1). The groups treated with Compound 3 exhibited a dose-dependent reduction of auranofin-induced p38 phosphorylation. The normalized levels of phosphorylated p38 in the group treated with 10 mg/kg Compound 3 was similar to those of the control group (FIG. 1B; * p<0.05 vs. vehicle; #p<0.05 vs. auranofin using the unpaired t-test).

### Example 2

### Characterization of ASK1 Inhibitor in the Sugen/Hypoxia Model of Pulmonary Hypertension

In the Sugen/hypoxia (Su/Hx) model of pulmonary hypertension (PH), Sprague-Dawley rats were given Sugen-5416 (Semaxanib; 200 mg/kg, subcutaneous) and housed in a hypoxic chamber (maintained at approximately ≦13% oxygen) to induce PH. The sham control rats received an injection of saline and were housed under normoxic conditions. The Su/Hx rats were administered with vehicle, Compound 4, or sildenafil for 4 weeks. Compound 4 was given in chow (0.1% or 0.2% as diet administered by weight) for 4 weeks. Sildenafil was administered twice a day via oral gavage (60 mg/kg/day, oral).

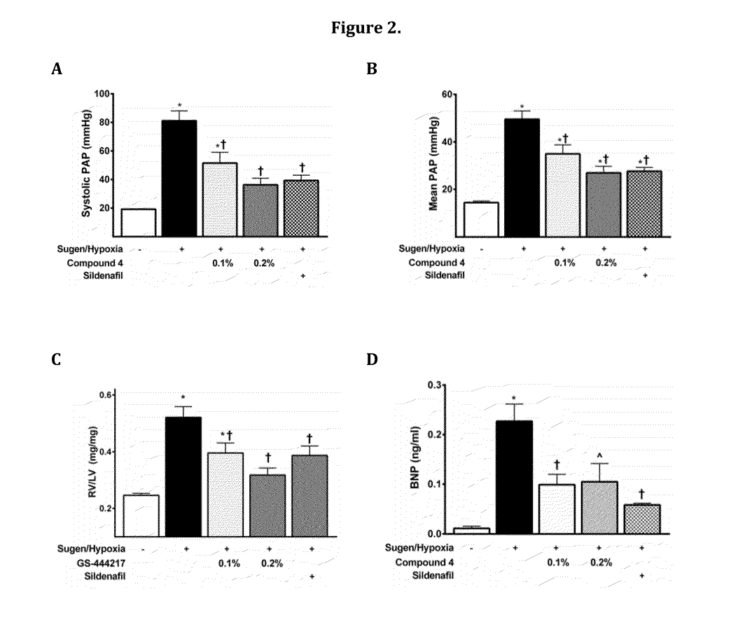

Four weeks after disease induction (i.e. Su/Hx treatment), the Su/Hx rats exhibited increased pulmonary arterial pressures (PAP) compared to those of the sham group, as measured by direct pulmonary arterial catheterization: systolic PAP was 79±21 (mean±SEM) vs. 19±1 mmHg, mean PAP was 49±11 vs. 15±1 mmHg, and diastolic PAP was 35±8 vs. 11±2 mmHg (all shown as Su/Hx vs. sham control). As shown in FIG. 2, the Su/Hx rats exhibited increased RV hypertrophy, compared to those of the sham group, as measured by right-ventricular weight normalized to the weight of the left ventricle (LV) and septum: RV:LV was 0.49±0.1 vs. 0.25±0.01 (mean±SEM) (shown as Su/Hx vs. sham control). The groups treated with Compound 4 at 0.1% or 0.2% in chow exhibited a dose-dependent reduction in systolic, mean, and diastolic PAP: systolic PAP were 52±22 and 36±13 mmHg (mean±SEM), mean PAP were 35±11 and 27±8 mmHg, and diastolic PAP were 26±7 and 20±5 mmHg, for 0.1% and 0.2% Compound 4 respectively. Also, the group treated with Compound 4 exhibited a dose-dependently reduction in RV hypertrophy: RV:LV were 0.39±0.1 and 0.35±0.11 for 0.1% and 0.2% respectively.

Circulating plasma levels of BNP is a clinically validated biomarker of RV failure. The BNP plasma levels were increased in the Su/Hx rats compared to those of the sham control: 0.23±0.1 vs. 0.1±0.01 ng/mL (mean±SEM) (shown as Su/Hx vs. sham control). Plasma levels of BNP were reduced by both doses of Compound 4: 0.1±0.05 for 0.1% and 0.1±0.1 ng/mL for 0.2% (FIG. 2D).

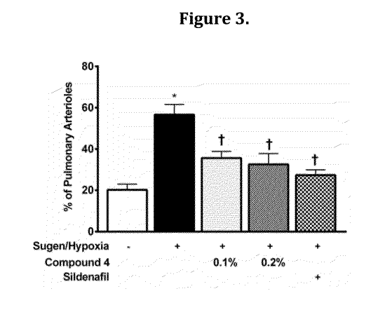

Muscularization of small peripheral pulmonary arteries was also characterized. Alpha-smooth muscle actin (α-SMA)/elastin-stained lung sections were categorized as nonmuscularized (exhibit elastin but no apparent smooth muscle), partially muscularized (incomplete medial layer of smooth muscle), or completely muscularized (concentric medial layer of smooth muscle). About 56.6% of arterioles were completely muscularized in the Su/Hx rats treated with vehicle. Su/Hx rats treated with 0.1% or 0.2% Compound 4 had a decreased number of completely muscularized arterioles (35.6% and 32.6%, respectively) (FIG. 3).

### Example 7

### Treating Subjects with PAH with ASK1 Inhibitor

In the in vivo model shown above, ASK1 inhibitor, such as the compounds having formula (I), was shown to be effective, e.g. reducing or improving pulmonary pressure, decreasing pulmonary vascular remodeling, and decreasing maladaptive RV hypertrophy.

In further studies, subjects with PAH receive placebo or a compound of formulae (I) or (IA) (2 mg, 6 mg, or 18 mg, once daily, orally) for a period of 24 weeks. Subjects are those having Group 1 PAH with a diagnosis of idiopathic PAH (IPAH), hereditary PAH (HPAH) or PAH associated with connective tissue disease (PAH-CTD), congenital heart defects, drug and toxin use, or human immunodeficiency virus (HIV) infection.

The study monitors several variables, including the change from baseline in pulmonary vascular resistance (PVR) as measured by right heart catheterization, as well as the change from baseline in cardiac index (CI) mean pulmonary artery pressure (mPAP), mean right atrial pressure (mRAP), mixed venous oxygen saturation (SvO₂), and right ventricular cardiac power. Also, the study monitors the change from baseline in clinical measures of symptoms and function, including submaximal exercise (6-minute walk test (6MWT)), heart rate recovery (HRR) after the 6MWT, the Borg dyspnea index, World Health Organization (WHO) Functional Class, N-terminal pro-brain natriuretic peptide, and/or quality of life by the SF-36® Health Survey.

What is claimed is:

1. A method of treating and/or preventing pulmonary vascular disease and/or right ventricle dysfunction in a patient in need thereof, comprising administering to the patient a therapeutically effective amount of an ASK1 inhibitor.

2. The method of claim 1, wherein the ASK1 inhibitor is a compound of formula (I): 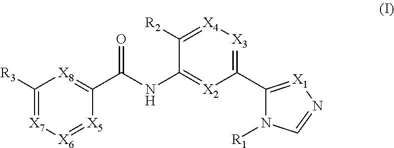
wherein:
R¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to three substituents selected from halo, oxo, alkyl, cycloalkyl, heterocyclyl, aryl, aryloxy, —NO₂, R⁶, —C(O)—R⁶, —OC(O)—R⁶—C(O)—O—R⁶, C(O)—N(R⁶)(R⁷), —OC(O)—N(R⁶)(R⁷), —S—R⁶, —S(═O)—R⁶, —S(═O)₂R⁶, —S(═O)₂—N(R⁶)(R⁷), —S(═O)₂—O—R⁶, —N(R⁶)(R⁷), —N(R⁶)—C(O)—R⁷, —N(R⁶)—C(O)—O—R⁷, —N(R⁶)—C(O)—N(R⁶)(R⁷), —N(R⁶)—S(═O)₂—R⁶, —CN, and —O—R⁶, and wherein the alkyl, cycloalkyl, heterocyclyl, phenyl, and phenoxy are optionally substituted by from one to three substituents selected from alkyl, cycloalkyl, alkoxy, hydroxyl, and halo; wherein R⁶ and R⁷ are independently selected from the group consisting of hydrogen, C₁-C₁₅ alkyl, cycloalkyl, heterocyclyl, aryl, and heteroaryl, all of which are optionally substituted with from one to three substituents selected from halo, alkyl, monoalkylamino, dialkylamino, alkyl amide, aryl amide, heteroaryl amide, —CN, lower alkoxy, —CF₃, aryl, and heteroaryl; or
R⁶ and R⁷ when taken together with the nitrogen to which they are attached form a heterocycle;
R² is hydrogen, halo, cyano, alkoxy, or alkyl optionally substituted by halo;
R³ is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —O—C(O)—R⁶, —O—C(O)—N(R⁶)(R⁷), —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—R⁶, —C(O)—N(R⁶)(R⁷), and —N(R⁶)—S(═O)₂—R⁷, wherein the alkyl, alkoxy, cycloalkyl, aryl, heteroaryl or heterocyclyl is optionally substituted with from one to five substituents selected from halo, oxo, —NO₂, alkyl, haloalkyl, haloalkoxy, —N(R⁶)(R⁷), —C(O)—R⁶, —C(O)—O—R⁶, —C(O)—N(R⁶)(R⁷), —CN, —O—R⁶, cycloalkyl, aryl, heteroaryl and heterocyclyl; with the proviso that the heteroaryl or heterocyclyl moiety includes at least one ring nitrogen atom;
X¹, X², X³, X⁴, X⁵, X⁶, X⁷ and X⁸ are independently C(R⁴) or N, in which each R⁴ is independently hydrogen, alkyl, alkoxy, cycloalkyl, aryl, heteroaryl, heterocyclyl, halo, —NO₂, haloalkyl, haloalkoxy, —CN, —O—R⁶, —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—O—R⁶, —C(O)—N(R⁶)(R⁷), or —N(R⁶)—S(═O)₂—R⁷, wherein the alkyl, cycloalkyl, aryl, heteroaryl, and heterocyclyl is further optionally substituted with from one to five substituents selected from halo, oxo, —NO₂, —CF₃, —O—CF₃, —N(R⁶)(R⁷), —C(O)—R⁶, —C(O)—O—R⁷, —C(O)—N(R⁶)(R⁷), —CN, —O—R⁶; or
X⁵ and X⁶ or X⁶ and X⁷ are joined to provide optionally substituted fused aryl or optionally substituted fused heteroaryl; and
with the proviso that at least one of X², X³, and X⁴ is C(R⁴); at least two of X⁵, X⁶, X⁷, and X⁸ are C(R⁴); and at least one of X², X³, X⁴, X⁵, X⁶, X⁷ and X⁸ is N;
or a pharmaceutically acceptable salt, isomer, or a mixture thereof.

3. The method of any of claim 1, wherein the ASK1 inhibitor is a compound selected from the group consisting of 3-(4-cyclopropyl-1H-imidazol-1-yl)-N-(6-(4-isopropyl-4H-1,2,4-triazol-3-yl)pyridin-2-yl)benzamide, 3-(4-cyclopropyl-1H-imidazol-1-yl)-N-(6-(4-cyclopropyl-4H-1,2,4-triazol-3-yl)pyridin-2-yl)-4-methylbenzamide, 5-(4-cyclopropyl-1 H-imidazol-1-yl)-2-fluoro-N-(6-(4-isopropyl-4H-1,2,4-triazol-3-yl)pyridin-2-yl)-4-methylbenzamide, 4-(4-cyclopropyl-1H-imidazol-1-yl)-N-(3-(4-cyclopropyl-4H-1,2,4-triazol-3-yl)phenyl)picolinamide, and (S)-5-(4-cyclopropyl-1H-imidazol-1-yl)-2-fluoro-4-methyl-N-(6-(4-(1,1,1-trifluoropropan-2-yl)-4H-1,2,4-triazol-3-yl)pyridin-2-yl)benzamide, or a pharmaceutically acceptable salt thereof.

4. The method of claim 1, wherein the ASK1 inhibitor is a compound of formula (II): 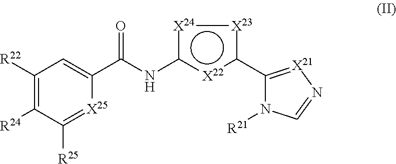
wherein:
R²¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to four substituents selected from the group consisting of halo, hydroxyl, oxo, alkyl, cycloalkyl, heterocyclyl, aryl, aryloxy, NO₂, R²⁶, C(O)R²⁶, OC(O)R²⁶C(O)OR²⁶, C(O)N(R²⁶)(R²⁷), OC(O)N(R²⁶)(R²⁷), SR²⁶, S(═O)R²⁶, S(═O)₂R²⁶, S(═O)₂N(R²⁶)(R²⁷), S(═O)₂OR²⁶, N(R²⁶)(R²⁷), N(R²⁶)C(O)R²⁷, N(R²⁶)C(O)OR²⁷, N(R²⁶)C(O)N(R²⁶)(R²⁷), N(R²⁶)S(═O)₂R²⁶, CN, and OR²⁶, wherein the alkyl, cycloalkyl, heterocyclyl, aryl, and aryloxy are optionally substituted with from one to three substituents selected from alkyl, cycloalkyl, alkoxy, hydroxyl, and halo;
R²⁶ and R²⁷ are independently selected from the group consisting of hydrogen, alkyl, cycloalkyl, heterocyclyl, aryl, and heteroaryl, wherein the alkyl, cycloalkyl, heterocyclyl, aryl, and heteroaryl are optionally substituted with from one to three substituents selected from halo, alkyl, monoalkylamino, dialkylamino, alkyl amide, aryl amide, heteroaryl amide, CN, lower alkoxy, CF₃, aryl, and heteroaryl; or
R²⁶ and R²⁷ when taken together with the nitrogen to which they are attached form a heterocycle;
R²² is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, NO₂, haloalkyl, haloalkoxy, CN, OR²⁶, OC(O)R²⁶, OC(O)N(R²⁶)(R²⁷), SR²⁶, N(R²⁶)(R²⁷), S(═O)R²⁶, S(═O)₂R²⁶, S(═O)₂N(R²⁶)(R²⁷), S(═O)₂OR²⁶, N(R²⁶)C(O)R²⁷, N(R²⁶)C(O)OR²⁷, N(R²⁶)C(O)N(R²⁶)(R²⁷), C(O)R²⁶, C(O)OR²⁶, C(O)N(R²⁶)(R²⁷), and N(R²⁶)S(═O)₂R²⁷ and wherein the alkyl, alkoxy, cycloalkyl, aryl, heteroaryl and heterocyclyl are optionally substituted with one or more substituents selected from halo, oxo, NO₂, alkyl, haloalkyl, haloalkoxy, N(R²⁶)(R²⁷), C(O)R²⁶, C(O)OR²⁶, C(O)N(R²⁶)(R²⁷), CN, OR²⁶, cycloalkyl, aryl, heteroaryl and heterocyclyl; with the proviso that the heteroaryl or heterocyclyl moiety includes at least one ring nitrogen atom;
R²⁴ and R²⁵ are independently hydrogen, halo, cyano, alkyl, alkoxy, or cycloalkyl, wherein the alkyl, alkoxy, and cycloalkyl are optionally substituted by halo or cycloalkyl;
X²¹ and X²⁵ are independently C(R²³) or N, wherein each R²³ is independently hydrogen, halo, alkyl, alkoxy or cycloalkyl, wherein the alkyl and cycloalkyl are optionally substituted with from one to five substituents selected from halo, oxo, CF₃, OCF₃, N(R²⁶)(R²⁷), C(O)R²⁶, C(O)OR²⁷, C(O)N(R²⁶)(R²⁷), CN, and OR²⁶; and
X²², X²³ and X²⁴ are independently C(R²³), N, O, or S; with the proviso that at least one of X²², X²³, and X²⁴ is C(R²³); and only one of X²², X²³, and X²⁴ is O or S;
or a pharmaceutically acceptable salt, isomer, or a mixture thereof.

5. The method of claim 1, wherein the ASK1 inhibitor is a compound of formula (III): 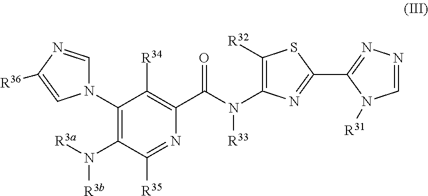
wherein:
R³¹ is C₁-C₃ alkyl or C₃-C₆ cycloalkyl, wherein the alkyl or cycloalkyl is optionally substituted with one to three halogen atoms;
R³² is hydrogen or C₁-C₆ alkyl wherein the alkyl is optionally substituted with halo.
R³³ is hydrogen or C₁-C₃ alkyl;
R³⁴ is hydrogen or C₁-C₃ alkyl;
R³⁵ is hydrogen, C₁-C₃ alkyl, OR^{3a} or —NHR^{3a};
R³⁶ is hydrogen, C₁-C₃ alkyl, C₁-C₃ haloalkyl, or C₃-C₆ cycloalkyl wherein the cycloalkyl is optionally substituted with C₁-C₃ alkyl, C₁-C₃ haloalkyl, or 1 or 2 halogen atoms;
R^{3a} and R^{3b} are independently hydrogen, C₁-C₃ alkyl or R^{3a} and R^{3b} combine with the nitrogen atom to which they are attached to form a four to six member heterocyclic ring optionally containing an oxygen or a nitrogen atom in the ring;
or a pharmaceutically acceptable salt, isomer, or mixture thereof.

6. The method of of claim 1, where in the pulmonary vascular disease is pulmonary hypertension.

7. The method of claim 1, wherein the pulmonary vascular disease is pulmonary arterial hypertension.

8. The method of claim 1, wherein the ASK1 inhibitor is administered at a dose of between 1 to 100 mg.

9. The method of claim 1, wherein the ASK1 inhibitor is administered at a dose of between 1 to 30 mg.

10. The method of claim 1, wherein the ASK1 inhibitor is administered orally, nasally, topically, or parenterally.

11. The method of claim 1, wherein the ASK1 inhibitor is administered daily.

12. The method of claim 1, wherein the ASK1 inhibitor is present in a pharmaceutical composition comprising the ASK1 inhibitor and at least one pharmaceutically acceptable carrier.

13. The method of claim 12, wherein the pharmaceutical composition is a tablet.

14. The method of claim 1, further comprising the administering of one or more therapeutic agent.

15. The method of claim 14, wherein the ASK1 inhibitor is administered sequentially with the one or more therapeutic agent.

16. The method of claim 14, wherein the ASK1 inhibitor is administered concurrently with the one or more therapeutic agent.

17. The method of claim 16, wherein the ASK1 inhibitor and one or more therapeutic agent is administered together in a single pharmaceutical composition.

18. A method of treating and/or preventing right ventricle failure, treating and/or preventing narrowing or restricting pulmonary arteries, or treating or improving PAH symptoms comprising administering an effective amount of ASK1 inhibitor.

19. A pharmaceutical composition comprising a therapeutically effective amount of an ASK1 inhibitor and at least one pharmaceutically acceptable carrier.

20. A kit comprises the pharmaceutical composition of claim 18 and a label and/or instructions for use.
